(19) United States
(12) Patent Application Publication
(10) Pub. No.: US 2026/0228844 A1
(43) Pub. Date: Aug. 6, 2026

(54) SYSTEMS AND METHODS FOR INTEGRATED HEALTHCARE MANAGEMENT WITH SOCIAL SERVICES

(71) Applicant: Latoya Coley, St. Petersburg, FL (US)

(72) Inventor: Latoya Coley, St. Petersburg, FL (US)

(21) Appl. No.: 19/532,399

(22) Filed: Feb. 6, 2026

Related U.S. Application Data
(60) Provisional application No. 63/755,066, filed on Feb. 6, 2025.

(51) Int. Cl. G06Q 50/22; G16H 10/60; G16H 20/70; G16H 50/30

(52) U.S. Cl. CPC G06Q 50/22; G16H 10/60; G16H 20/70; G16H 50/30

(57) ABSTRACT

A computer-implemented care coordination system integrates healthcare data, shelter management data, and social determinants of health data into a unified platform. The system exchanges clinical data with external electronic health record systems through Fast Healthcare Interoperability Resources based interfaces, enabling bidirectional synchronization of medical history and mental health diagnosis information. A shelter management module tracks bed availability, intake status, and food service availability in real-time from shelter facilities. A social determinants of health module collects housing status, employment status, and food access metrics through mobile devices or caseworker entry. A predictive analytics engine analyzes combined data from all three domains to generate risk assessments that identify individuals at elevated risk for mental health crises or hospital readmissions. The system generates intervention recommendations including caseworker outreach notifications, mental health service referrals, and healthcare provider alerts, and maintains audit records documenting all data access and modifications.

### CROSS-REFERENCE TO RELATED APPLICATIONS

The present application claims priority to U.S. Provisional Application No. 63/755,066 filed Feb. 6, 2025, titled “SYSTEMS AND METHODS FOR INTEGRATED HEALTHCARE MANAGEMENT WITH SOCIAL SERVICES,” which is hereby incorporated by reference in its entirety.

### TECHNICAL FIELD

The embodiments generally relate to the technical field of computer-based healthcare coordination and social services management systems for integrating electronic health records, shelter resources, and social determinants of health data.

### BACKGROUND

Conventional healthcare management systems are designed to facilitate documentation and tracking of clinical data for patient populations. Such systems often operate as electronic health record (EHR) platforms that store medical histories, diagnoses, treatment plans, and other clinical information within healthcare provider networks.

These systems typically support the storage and organization of patient health records, appointment scheduling, and clinical workflow management. Many employ standardized data formats and application programming interfaces to enable data exchange between healthcare providers within integrated health networks. EHR platforms such as Epic, Cerner, and similar systems have become widely adopted across hospitals, clinics, and healthcare organizations for managing clinical documentation and facilitating care delivery.

Conventional solutions may also generate reports or analytics summarizing patient outcomes, resource utilization, and population health metrics. These reports are often used by healthcare administrators to assess operational performance and allocate resources. In many cases, healthcare data within these systems remains focused on clinical encounters and medical interventions, with limited integration of non-clinical factors that influence patient health outcomes.

Separately, shelter management systems exist to track bed availability, intake processes, and service offerings at homeless shelters and transitional housing facilities. These systems typically operate independently from healthcare platforms, requiring shelter staff to manually coordinate with healthcare providers when residents require medical attention. Social service agencies may maintain additional databases tracking employment assistance, food access programs, and housing placement services, often without connectivity to either healthcare or shelter systems.

While such systems provide efficiencies within their respective domains, they face challenges in coordinating care across healthcare, shelter, and social service boundaries. Healthcare providers treating homeless individuals often lack visibility into shelter status, housing history, or social service engagement. Shelter staff may be unaware of residents'medical conditions, mental health diagnoses, or upcoming healthcare appointments. Caseworkers coordinating social services may not have access to clinical information that could inform intervention strategies. This fragmentation leads to duplicated data entry efforts, missed opportunities for early intervention, and difficulty identifying individuals at elevated risk for adverse outcomes.

Consequently, there is a need for an improved care coordination system that addresses the limitations of siloed healthcare, shelter, and social service platforms, reduces reliance on manual coordination processes, and provides enhanced integration between clinical data, shelter resources, and social determinants of health tracking.

### SUMMARY

This summary is provided to introduce a variety of concepts in a simplified form that is further disclosed in the detailed description of the embodiments. This summary is not intended to identify key or essential inventive concepts of the claimed subject matter, nor is it intended to determine the scope of the claimed subject matter.

In one aspect, the disclosed system, method, or software product may include a centralized database configured to store healthcare data, shelter management data, and social determinants of health data for a plurality of individuals. The database may serve as a unified repository that enables data from multiple domains to be accessed, analyzed, and coordinated through a single platform.

In one aspect, embodiments may include an electronic health record interface module configured to exchange healthcare data with external electronic health record systems via application programming interfaces. The module may retrieve clinical data such as medical history and mental health diagnoses from external EHR systems and transmit updated data back to those systems, enabling bidirectional data flow between the care coordination platform and existing healthcare infrastructure.

In one aspect, embodiments may include a shelter management module configured to track shelter resource data comprising bed availability and intake status for shelter facilities. The module may communicate shelter resource data to the centralized database for integration with healthcare data, enabling healthcare providers and caseworkers to access shelter status information alongside clinical records.

In one aspect, embodiments may include a social determinants of health module configured to collect and store social determinants of health data comprising housing status, employment status, and food access metrics for individuals. The module may receive data via mobile device input or caseworker data entry, enabling field-based collection of non-clinical factors that influence health outcomes.

In one aspect, embodiments may include a predictive analytics engine configured to analyze a combined dataset comprising healthcare data, shelter management data, and social determinants of health data to generate risk assessments identifying at-risk individuals. The engine may apply algorithms to integrated data from multiple domains to predict risks such as mental health crises or hospital readmissions, enabling proactive interventions before adverse events occur.

In one aspect, embodiments may include a user interface module configured to provide access to the care coordination platform for a plurality of user types comprising healthcare providers, shelter staff, and caseworkers. The module may display customizable dashboards presenting healthcare data, shelter management data, and social determinants of health data, and may support mobile access via mobile computing devices to enable field-based interaction with the platform.

In some aspects, the system may include at least one computing device in operable communication with a network and a server in operable communication with the network to host a care coordination platform containing the above-described modules. The computing device may execute instructions to perform the operations described herein, thereby enabling integrated, data-driven care coordination that improves outcomes for individuals requiring healthcare services, shelter resources, and social services support.

Other illustrative variations within the scope of the invention will become apparent from the detailed description provided hereinafter. The detailed description and enumerated variations, while disclosing optional variations, are intended for purposes of illustration only and are not intended to limit the scope of the invention.

### BRIEF DESCRIPTION OF THE DRAWINGS

A more complete understanding of the embodiments, and the attendant advantages and features thereof, will be more readily understood by references to the following detailed description when considered in conjunction with the accompanying drawings wherein:

FIG. 1 illustrates a system architecture diagram, according to some embodiments;

FIG. 2 illustrates an application program and modules in communication with the computing system, according to some embodiments;

FIG. 3 is a flow diagram illustrating an exemplary operational sequence of the Electronic Health Record Interface Module, according to some embodiments;

FIG. 4 is a flow diagram illustrating an exemplary operational sequence of the Shelter Management Module, according to some embodiments;

FIG. 5 is a flow diagram illustrating an exemplary operational sequence of the Social Determinants of Health Module, according to some embodiments;

FIG. 6 is a flow diagram illustrating an exemplary operational sequence of the Predictive Analytics Engine, according to some embodiments; and

FIG. 7 is a flow diagram illustrating an exemplary end-to-end system operational flow, according to some embodiments.

### DETAILED DESCRIPTION

The specific details of the single embodiment or variety of embodiments described herein are set forth in this application. Any specific details of the embodiments described herein are used for demonstration purposes only, and no unnecessary limitation(s) or inference(s) are to be understood or imputed therefrom.

Before describing exemplary embodiments in detail, it is noted that the embodiments reside primarily in combinations of components related to devices and systems. Accordingly, the device components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

The disclosed system may include at least one computing device in operable communication with a network and a server configured to host and execute a care coordination platform. The care coordination platform may include multiple functional modules, each implemented in software, firmware, hardware, or any combination thereof. In some embodiments, the modules may include an Electronic Health Record Interface Module configured to exchange healthcare data with external electronic health record systems via application programming interfaces, including retrieving clinical data and transmitting updated data bidirectionally. A Shelter Management Module may track shelter resource data comprising bed availability, intake status, and food service availability for shelter facilities, and may communicate shelter resource data to a centralized database for integration with healthcare data. A Social Determinants of Health Module may collect and store social determinants of health data comprising housing status, employment status, and food access metrics for individuals via mobile device input or caseworker data entry. A Predictive Analytics Engine may analyze a combined dataset comprising healthcare data, shelter management data, and social determinants of health data to generate risk assessments identifying at-risk individuals and intervention recommendations. A Communication and User Interface Module may provide access to the care coordination platform for a plurality of user types comprising healthcare providers, shelter staff, and caseworkers, and may display customizable dashboards via mobile devices or web browsers. An Audit Module may generate records documenting data access and data modifications by each user type to create verifiable audit trails. A Database Engine may manage structured data storage and retrieval operations for the Data Repository.

Conventional healthcare management platforms often process patient data in isolated silos, with limited ability to integrate clinical information with shelter resources or social service data in real-time or coordinate care across organizational boundaries automatically. Electronic health record systems typically focus on clinical encounters and medical interventions without visibility into housing status, shelter utilization, or social service engagement. Shelter management systems operate independently, requiring manual coordination when residents require healthcare services. Social service agencies maintain separate databases tracking employment assistance, food access programs, and housing placement without connectivity to healthcare or shelter systems. The disclosed embodiments address this problem by combining bidirectional electronic health record integration with real-time shelter resource tracking, social determinants of health data collection, and predictive analytics to enable coordinated care across traditionally fragmented service domains. This configuration enables automated, data-driven care coordination that can dynamically identify at-risk individuals based on integrated data from multiple sources while maintaining complete audit trails.

In practice and in use, the system may be deployed by healthcare organizations, shelter networks, or social service agencies that serve individuals requiring coordinated support across healthcare, housing, and social services. When a healthcare provider, shelter staff member, or caseworker accesses the care coordination platform, the Electronic Health Record Interface Module may retrieve clinical data from external electronic health record systems such as Epic or other EHR platforms via Fast Healthcare Interoperability Resources (FHIR)-based application programming interfaces. The Shelter Management Module may track real-time bed availability and intake status from shelter facility systems. The Social Determinants of Health Module may collect data on housing status, employment status, and food access metrics through mobile device input or caseworker data entry. The Predictive Analytics Engine may analyze the combined dataset to generate risk assessments identifying individuals at elevated risk for mental health crises or hospital readmissions. Based on the risk assessment, the system may generate intervention recommendations comprising caseworker outreach notifications, mental health service referrals, or healthcare provider alerts. If an individual is identified as at-risk, the appropriate user types may receive notifications through the Communication and User Interface Module to enable proactive intervention before adverse events occur.

In this way, the system may improve care coordination by automating the integration of healthcare, shelter, and social service data, reducing reliance on manual coordination processes, and enabling proactive interventions through predictive analytics. The real-time electronic health record integration eliminates duplicate data entry requirements and ensures that clinical information is current across all connected systems. The shelter management functionality provides healthcare providers and caseworkers with visibility into housing status and shelter utilization that was previously unavailable without manual phone calls or site visits. The social determinants of health tracking enables systematic collection of non-clinical factors that influence health outcomes, allowing care teams to address root causes rather than treating symptoms in isolation. The Predictive Analytics Engine identifies at-risk individuals before crises occur, enabling proactive outreach rather than reactive emergency responses. The Audit Module preserves detailed records of data access and modifications, as such, the system provides verifiable documentation of care coordination activities for regulatory review and quality improvement. These capabilities can reduce emergency room visits by enabling early intervention, improve health outcomes by addressing social determinants alongside clinical needs, and optimize resource utilization by coordinating services across organizational boundaries.

Various implementations of the present disclosure involve the technical field of computer-based healthcare coordination and social services management systems for integrating electronic health records, shelter resources, and social determinants of health data, including executing algorithms to exchange clinical data bidirectionally with external EHR systems, integrating real-time shelter resource data into care coordination workflows, applying predictive analytics to combined datasets from multiple domains, generating risk assessments and intervention recommendations based on integrated data analysis, and synchronizing structured audit records across distributed care coordination systems. These operations are inherently computer-based and cannot be performed in the human mind or using pen and paper due to the volume, speed, and complexity of the data being processed. For example, the system executes the steps of receiving clinical data from multiple external EHR systems via FHIR-based APIs, tracking real-time shelter resource availability across multiple facilities, collecting and parsing social determinants of health data from mobile devices and caseworker entries, analyzing combined datasets comprising thousands of data points across healthcare, shelter, and social service domains, and generating risk assessments and intervention recommendations in real-time based on integrated data from multiple sources. The present disclosure amounts to more than merely implementing a generic computer as a tool to gather, analyze, and output data because the claimed operations improve the field of healthcare coordination by enabling automated, data-driven integration that actively identifies at-risk individuals and generates intervention recommendations rather than merely documenting service delivery after the fact. In particular, the speed at which the steps of the present disclosure occur to effectuate the disclosed method, system, or product would involve real-time processing of clinical data from external EHR systems, instantaneous integration of shelter resource data across distributed facilities, and immediate generation of risk assessments when predictive analytics identify elevated risk levels. That is, the steps of the present method, system, or product are impossible to accomplish on pen and paper, cannot be accomplished as a method of organizing human activity, and amount to more than merely gathering, analyzing, and outputting data.

Various implementations of the present disclosure include executing computer-implemented data exchange algorithms, predictive analytics routines, and risk assessment engines on computing hardware to coordinate care across healthcare, shelter, and social service domains in real-time. The computing system implements these algorithms when it performs tasks such as parsing clinical data received from external EHR systems via FHIR-based APIs, tracking shelter resource availability in real-time across multiple facilities, collecting and storing social determinants of health data from mobile inputs, analyzing combined datasets using predictive analytics algorithms, generating risk assessments identifying at-risk individuals, and producing intervention recommendations based on integrated analysis results. In particular, the speed at which the system processes real-time data from external EHR systems, integrates shelter resource data from multiple facilities, analyzes combined datasets comprising healthcare, shelter, and social determinants of health data, and generates risk assessments and intervention recommendations would involve continuous, high-frequency data processing across networked systems. As such, the present disclosure would be impossible to accomplish on pen and paper or in the human mind due to the volume of real-time data being processed from multiple external systems, the number of simultaneous data integration operations being performed, and the speed required to identify at-risk individuals and generate intervention recommendations before adverse events occur.

In some embodiments, the Electronic Health Record Interface Module processes data exchange requests by establishing connections with external EHR systems via FHIR-based application programming interfaces, transmitting data requests, receiving clinical data comprising medical history and mental health diagnosis data, and transmitting updated data back to external EHR systems. The Shelter Management Module retrieves bed availability data, intake status data, and food service availability data from shelter facility systems, communicates shelter resource data to the centralized database, and integrates shelter resource data with healthcare data. The Social Determinants of Health Module receives data input via mobile devices or caseworker data entry, parses received data to extract housing status, employment status, and food access metrics, associates parsed data with individual records, and stores social determinants of health data in the centralized database. The Predictive Analytics Engine retrieves healthcare data, shelter management data, and social determinants of health data from the centralized database, combines retrieved data into a combined dataset, analyzes the combined dataset using predictive analytics algorithms, generates risk assessments identifying at-risk individuals, determines risk types comprising mental health crisis risk or hospital readmission risk, and generates intervention recommendations based on the risk assessment. The foregoing operations are executed as sequences of API calls, database queries, algorithmic analyses, conditional logic evaluations, and network communications across distributed systems, and cannot practically be performed in the human mind or on paper. These concrete processing steps of bidirectional EHR data exchange, real-time shelter resource integration, social determinants of health data collection, predictive analytics on combined datasets, and intervention recommendation generation provide a specific improvement to healthcare coordination systems by enabling proactive identification of at-risk individuals through automated data integration mechanisms, rather than a mere abstract idea of organizing human activity.

The disclosed care coordination platform incorporates several technical features that distinguish it from conventional healthcare management systems. In some embodiments, the platform may implement bidirectional electronic health record integration that exchanges clinical data with external EHR systems in real-time via standardized application programming interfaces. Unlike conventional systems that maintain healthcare data in isolated repositories, the disclosed platform may retrieve clinical data such as medical history and mental health diagnoses from external EHR systems and may transmit updated data back to those systems to maintain synchronization across connected platforms. The Electronic Health Record Interface Module may utilize FHIR-based APIs to enable interoperability with EHR platforms such as Epic and other commercially available systems, ensuring that clinical information flows seamlessly between the care coordination platform and existing healthcare infrastructure without requiring manual data entry or duplicate record maintenance.

In some embodiments, the platform may implement real-time shelter resource tracking that monitors bed availability, intake status, and food service availability across shelter facilities. Unlike conventional systems where shelter data remains isolated from healthcare systems, the disclosed platform may integrate shelter resource data directly with healthcare data in the centralized database. When shelter resource data changes, such as when beds become available or intake status is updated, the Shelter Management Module may communicate updated data to the centralized database in real-time, enabling healthcare providers and caseworkers to access current shelter information alongside clinical records. This integration may enable care teams to coordinate housing placement with healthcare discharge planning, identify shelter utilization patterns that correlate with health outcomes, and allocate shelter resources based on clinical needs.

In some embodiments, the platform may implement systematic social determinants of health data collection that captures housing status, employment status, and food access metrics for individuals through mobile device input or caseworker data entry. Unlike conventional systems that focus exclusively on clinical encounters, the disclosed platform may collect and store non-clinical factors that influence health outcomes in the same centralized database as healthcare and shelter data. The Social Determinants of Health Module may receive data from field-based caseworkers using mobile devices, parse received data to extract specific metrics, and associate collected data with individual records to build comprehensive profiles that span clinical, housing, and social service domains. In some embodiments, the social determinants of health data may further comprise transportation access, income level, or education status to capture additional factors that influence health outcomes and service needs.

In some embodiments, the platform may implement predictive analytics that analyze combined datasets to generate risk assessments identifying at-risk individuals before adverse events occur. Unlike conventional systems that generate reports summarizing historical service delivery, the disclosed platform may apply predictive analytics algorithms to integrated data from healthcare, shelter, and social service domains to identify individuals at elevated risk for future adverse events. The Predictive Analytics Engine may analyze patterns across the combined dataset to determine risk types comprising mental health crisis risk or hospital readmission risk, enabling proactive intervention rather than reactive crisis response. When the risk assessment identifies an at-risk individual, the system may generate intervention recommendations comprising caseworker outreach notifications, mental health service referrals, or healthcare provider alerts, directing appropriate resources to address identified risks before they escalate to emergencies.

In some embodiments, the platform may implement role-based access that provides customized interfaces for different user types comprising healthcare providers, shelter staff, and caseworkers. The Communication and User Interface Module may generate user interfaces that display relevant information based on user type, enabling each user to access the data and functions appropriate to their role in the care coordination process. Healthcare providers may view clinical data alongside shelter status and social determinants of health metrics. Shelter staff may view bed availability and intake information alongside relevant health information for residents. Caseworkers may view comprehensive profiles spanning healthcare, shelter, and social service domains to coordinate holistic support for individuals. In some embodiments, the user interface module may be configured to display a customizable dashboard presenting healthcare data, shelter management data, and social determinants of health data in consolidated views that enable users to quickly assess individual needs and service utilization.

In some embodiments, the platform may implement mobile access that enables field-based interaction with the care coordination platform via mobile computing devices. Unlike conventional systems that require access to fixed workstations, the disclosed platform may provide mobile access through the Communication and User Interface Module, enabling caseworkers to collect social determinants of health data during field visits, shelter staff to update resource availability from any location within facilities, and healthcare providers to access care coordination information during patient encounters. This mobile accessibility may enable real-time data collection and retrieval in settings where fixed computing infrastructure is unavailable or impractical, extending the reach of the care coordination platform to locations where services are actually delivered.

In some embodiments, the platform may implement cloud-based data synchronization that maintains consistent information across a plurality of shelter facilities and service locations. The server may comprise a cloud-based server configured to synchronize healthcare data, shelter management data, social determinants of health data, and audit records across distributed facilities in real-time. This cloud-based architecture may enable organizations operating multiple shelter locations, healthcare facilities, or service sites to maintain unified records without requiring manual data transfer between locations. When data is updated at any connected facility, the cloud-based synchronization may propagate changes to the centralized database, ensuring that all users across all locations access current information regardless of where data was originally entered.

In some embodiments, the platform may implement audit trail generation that documents data access and data modifications by each user type to create verifiable records of care coordination activities. The Audit Module may record user identifiers, timestamps, data accessed, and modifications made throughout care coordination workflows. This comprehensive documentation may support regulatory compliance requirements, quality improvement initiatives, and accountability for data handling across organizational boundaries. The audit trail may enable administrators to verify that appropriate users accessed appropriate data, track the provenance of information used in care coordination decisions, and demonstrate compliance with data protection and privacy requirements.

These technical features may collectively transform care coordination from a fragmented, manual process into an integrated, automated system. Whereas conventional approaches require separate interactions with healthcare systems, shelter management systems, and social service databases, the disclosed platform may integrate data from all three domains into a unified repository that enables comprehensive analysis and coordinated intervention. By combining bidirectional EHR integration, real-time shelter resource tracking, social determinants of health data collection, predictive analytics, and audit trail generation into a unified care coordination platform, the system may address fundamental limitations in current approaches that separate healthcare delivery from housing support and social services.

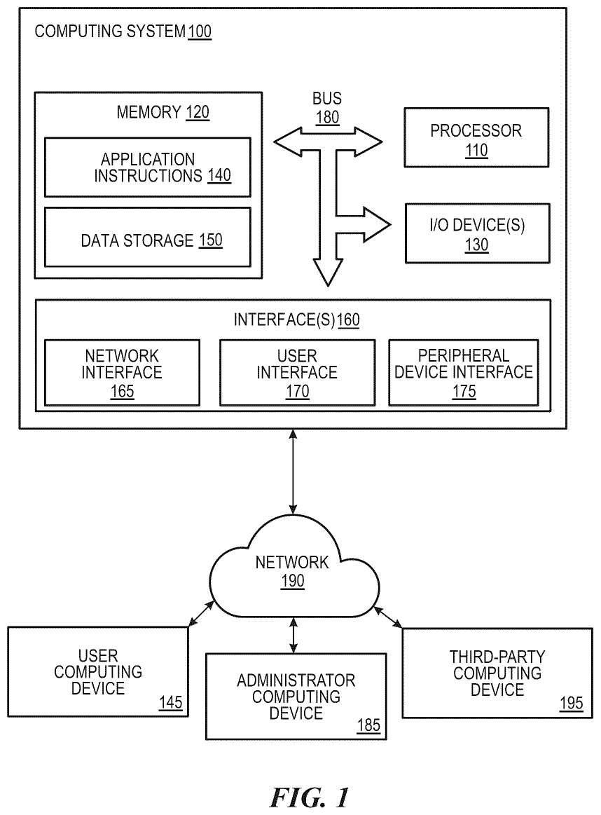

FIG. 1 illustrates an example of a computing system 100 that may provide the execution environment for implementing the processes and methods described herein. The computing system 100 may take various forms depending on deployment context, including but not limited to: a desktop or laptop computer, a tablet or smartphone, a server in a data center, a network appliance, a mainframe computer, a workstation, or a cloud-hosted virtual machine. In some embodiments, the computing system 100 may correspond to a distributed computing environment, such as a cluster of servers executing containerized workloads (e.g., Docker, Kubernetes), or an edge device integrated into Internet of Things (IoT) environments. In other embodiments, the computing system 100 may be embedded in another device, such as a vehicle infotainment unit, a medical diagnostic machine, an industrial robot controller, or a wearable computing device.

The computing system 100 includes one or more processors 110 operably coupled to a memory 120 via a system bus 180. The processor 110 may be implemented as a general-purpose central processing unit (CPU), a graphics processing unit (GPU), a tensor processing unit (TPU), a digital signal processor (DSP), or any combination thereof. In some embodiments, the processor 110 may be an application-specific integrated circuit (ASIC) optimized for a particular workload, a field-programmable gate array (FPGA), or a quantum or neuromorphic processor in advanced implementations. The processor 110 may include single-core, multi-core, or many-core configurations and may support hardware virtualization, multithreading, or parallel execution environments to optimize system performance.

The memory 120 may include volatile memory, nonvolatile memory, or a combination thereof. Volatile memory may include system RAM, cache memory, or high-bandwidth memory (HBM). Nonvolatile memory may include flash storage, solid-state drives (SSD), magnetic hard disk drives (HDD), optical storage devices, or persistent memory technologies such as Intel Optane. The memory 120 stores application instructions 140 for carrying out the functionalities described herein and data storage 150 for maintaining information related to system operations. The application instructions 140 may include code written in languages such as C, C++, Java, Python, Go, Rust, or JavaScript, as well as machine learning models trained using frameworks such as TensorFlow or PyTorch. The data storage 150 may contain structured information such as relational database records, unstructured data such as text or images, or real-time telemetry streams. In cloud-based embodiments, the memory 120 may represent scalable storage resources provisioned on-demand through Infrastructure-as-a-Service (IaaS) providers.

The computing system 100 may also include one or more input/output (I/O) devices 130. These devices may encompass visual output devices such as monitors, head-mounted displays, augmented reality (AR) glasses, or projectors; input devices such as keyboards, mice, touchscreens, styluses, or game controllers; and sensor devices such as microphones, cameras, depth sensors, biometric scanners, or environmental sensors. In industrial or medical environments, the I/O devices 130 may include robotic actuators, infusion pumps, or diagnostic imaging scanners. In vehicular environments, the I/O devices 130 may include in-cabin displays, steering sensors, and connected infotainment systems.

The computing system 100 further comprises one or more interfaces 160 that enable communication with other systems, users, or peripheral components. The network interface 165 allows the computing system 100 to exchange data with external systems across a network 190 using wired or wireless protocols. Example communication standards include Ethernet, Wi-Fi, Bluetooth, 5G, Long-Term Evolution (LTE), satellite communication, or emerging protocols such as Wi-Fi 7 or ultra-wideband (UWB). In some embodiments, the network interface 165 supports secure protocols such as HTTPS, TLS, or VPN tunneling to ensure authenticated and encrypted data transfer. The user interface 170 may include APIs, graphical user interfaces (GUIs), command-line interfaces (CLIs), or natural language interfaces enabled through speech recognition or chatbot systems. The peripheral device interface 175 enables connectivity with external hardware such as printers, external storage arrays, or specialized scientific equipment.

The network 190 represents any communication infrastructure capable of facilitating data exchange between computing entities. In some embodiments, the network 190 corresponds to a local area network (LAN) within a home or enterprise environment. In other embodiments, the network 190 may be a wide area network (WAN), a metropolitan area network (MAN), a peer-to-peer (P2P) communication mesh, or the global Internet. The network 190 may employ cloud orchestration layers, software-defined networking (SDN), or edge computing gateways. In high-security applications, the network 190 may implement firewalls, intrusion detection systems, or zero-trust architectures to protect transmitted data.

The computing system 100 is illustrated as being in communication with multiple external devices, including a user computing device 145, an administrator computing device 185, and a third-party computing device 195. The user computing device 145 may be a smartphone, tablet, laptop, or smart appliance configured to execute client-side applications or interact with system services. The administrator computing device 185 may be a workstation or remote management console configured to perform oversight functions such as monitoring, auditing, updating, or troubleshooting. The third-party computing device 195 may represent a partner system, vendor service, or external application interface that exchanges data with the computing system 100 via secure APIs. In cloud or SaaS embodiments, these devices may also include external microservices, data warehouses, or federated learning nodes.

In some embodiments, the computing system 100 may be deployed in a client-server model, where the computing system 100 acts as a backend server managing requests from client devices. In other embodiments, the computing system 100 may function within a cloud-native environment, operating as a microservice within a container orchestration platform. In edge deployments, the computing system 100 may be optimized for low-latency local processing, while synchronizing with centralized cloud infrastructure for data persistence and global coordination.

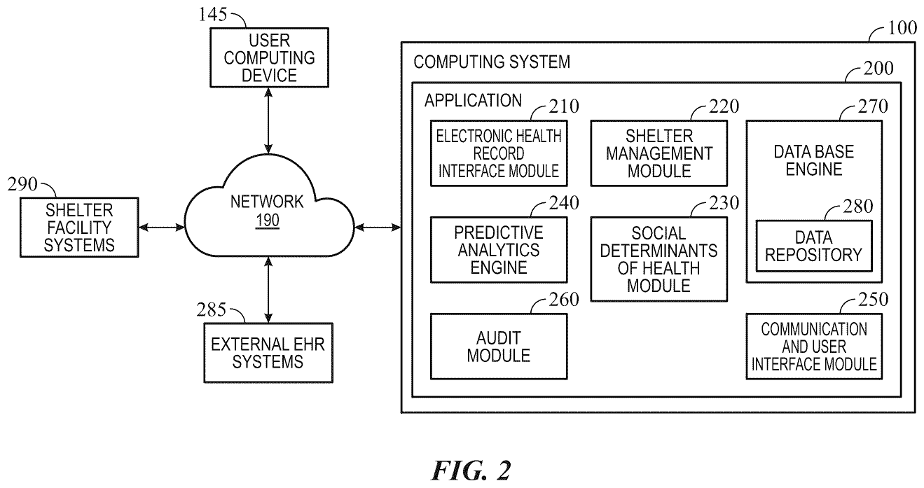

FIG. 2 illustrates an example computer architecture for the application program 200 operated via the computing system 100. The computing system 100 comprises several modules and engines configured to execute the functionalities of the application program 200, and a database engine 270 configured to facilitate how data is stored and managed in one or more databases. In particular, FIG. 2 is a block diagram showing the modules and engines needed to perform specific tasks within the application program 200.

Referring to FIG. 2, the computing system 100 operating the application program 200 comprises one or more modules having the necessary routines and data structures for performing specific tasks, and one or more engines configured to determine how the platform manages and manipulates data. In some embodiments, the application program 200 comprises one or more of an Electronic Health Record Interface Module 210, a Shelter Management Module 220, a Social Determinants of Health Module 230, a Predictive Analytics Engine 240, a Communication and User Interface Module 250, and an Audit Module 260. The application program 200 further interfaces with a Database Engine 270 that manages operations for the Data Repository 280. The computing system 100 communicates via network 190 with external systems including user computing devices 145, external electronic health record (EHR) systems 285, and shelter facility systems 290.

In some embodiments, the Electronic Health Record Interface Module 210 may be configured to exchange healthcare data with at least one external EHR system 285 via an application programming interface (API). The Electronic Health Record Interface Module 210 may receive data exchange requests from other components of the application program 200 and initiate communication sessions with external EHR systems 285 via the network 190. In some embodiments, the Electronic Health Record Interface Module 210 may establish connections using a Fast Healthcare Interoperability Resources (FHIR)-based API configured to enable bidirectional data exchange between the care coordination platform and the at least one external EHR system 285.

The Electronic Health Record Interface Module 210 may retrieve clinical data from the at least one external EHR system 285 by transmitting data requests formatted according to FHIR specifications, receiving response messages containing requested clinical data, and parsing received response messages to extract relevant data fields. In some embodiments, the clinical data retrieved by the Electronic Health Record Interface Module 210 may comprise at least one of medical history or mental health diagnosis data. The Electronic Health Record Interface Module 210 may parse received clinical data by identifying data elements within FHIR resource structures, extracting values from identified elements, and converting extracted values into internal data formats compatible with the Data Repository 280.

The Electronic Health Record Interface Module 210 may also transmit updated data to the at least one external EHR system 285 via the API. In some embodiments, the Electronic Health Record Interface Module 210 may format updated data according to FHIR resource specifications, establish secure connections with external EHR systems 285, transmit formatted data via HTTPS protocols, and receive confirmation responses indicating successful data transmission. This bidirectional data exchange may enable the care coordination platform to maintain synchronized records with external EHR systems 285 without requiring manual data entry or duplicate record maintenance. In some embodiments, the Electronic Health Record Interface Module 210 may be configured to exchange the healthcare data with the at least one external EHR system 285 in real-time by maintaining persistent connections or polling external systems at defined intervals.

In some embodiments, the Shelter Management Module 220 may be configured to track shelter resource data comprising at least bed availability and intake status for at least one shelter facility. The Shelter Management Module 220 may establish communication connections with shelter facility systems 290 via the network 190 using APIs, database connections, or other data exchange protocols. The Shelter Management Module 220 may retrieve bed availability data by querying shelter facility systems 290 for current occupancy counts, total bed capacity, and bed reservation status. In some embodiments, the Shelter Management Module 220 may retrieve intake status data by querying shelter facility systems 290 for intake queue lengths, intake processing times, and intake eligibility criteria.

The Shelter Management Module 220 may communicate the shelter resource data to the centralized database for integration with the healthcare data. In some embodiments, the Shelter Management Module 220 may format retrieved shelter resource data into structured records, transmit formatted records to the Database Engine 270, and store formatted records in the Data Repository 280 in association with corresponding individual records containing healthcare data. This integration may enable healthcare providers and caseworkers to access shelter status information alongside clinical records through a unified interface.

In some embodiments, the Shelter Management Module 220 may be configured to track the shelter resource data in real-time by receiving push notifications from shelter facility systems 290 when resource availability changes, or by polling shelter facility systems 290 at defined intervals to detect changes. When updated shelter resource data is received, the Shelter Management Module 220 may update corresponding records in the Data Repository 280 to reflect current conditions. In some embodiments, the Shelter Management Module 220 may be further configured to track food service availability for the at least one shelter facility by retrieving meal schedules, dietary accommodation options, and current food inventory status from shelter facility systems 290.

In some embodiments, the Social Determinants of Health Module 230 may be configured to collect and store social determinants of health (SDOH) data comprising at least housing status, employment status, and food access metrics for each of a plurality of individuals. The Social Determinants of Health Module 230 may receive SDOH data input via at least one of a mobile device input or a caseworker data entry. In some embodiments, the Social Determinants of Health Module 230 may generate data collection forms that are transmitted to user computing devices 145 via the Communication and User Interface Module 250, receive completed form data via the network 190, and parse received form data to extract individual data elements.

The Social Determinants of Health Module 230 may parse received SDOH data by identifying data fields within received form submissions, extracting values from identified fields, validating extracted values against defined data formats and value ranges, and converting validated values into internal data formats. In some embodiments, the Social Determinants of Health Module 230 may extract housing status by identifying housing-related fields indicating current living situation such as sheltered, unsheltered, transitional housing, or permanent housing. The Social Determinants of Health Module 230 may extract employment status by identifying employment-related fields indicating current work situation such as employed, unemployed, underemployed, or unable to work. The Social Determinants of Health Module 230 may extract food access metrics by identifying nutrition-related fields indicating food security status, access to food assistance programs, and frequency of adequate meals.

The Social Determinants of Health Module 230 may associate extracted SDOH data with individual records in the centralized database by matching received data to existing individual records based on unique identifiers, creating new individual records when no matching record exists, and linking extracted data elements to matched or created records. In some embodiments, the Social Determinants of Health Module 230 may store SDOH data in the Data Repository 280 via the Database Engine 270 by generating database insert or update commands, transmitting generated commands to the Database Engine 270, and receiving confirmation responses indicating successful storage operations.

In some embodiments, the SDOH data may further comprise at least one of transportation access, income level, or education status. The Social Determinants of Health Module 230 may collect transportation access data by identifying fields indicating availability of personal vehicles, access to public transportation, and transportation barriers to healthcare or employment. The Social Determinants of Health Module 230 may collect income level data by identifying fields indicating household income, income sources, and eligibility for income-based assistance programs. The Social Determinants of Health Module 230 may collect education status data by identifying fields indicating highest education level completed, current enrollment status, and educational barriers.

In some embodiments, the Predictive Analytics Engine 240 may be configured to analyze a combined dataset comprising the healthcare data, the shelter management data, and the SDOH data to generate a risk assessment identifying at-risk individuals from the plurality of individuals. The Predictive Analytics Engine 240 may retrieve healthcare data from the Data Repository 280 via the Database Engine 270 by generating database query commands specifying healthcare data tables and relevant selection criteria. The Predictive Analytics Engine 240 may retrieve shelter management data from the Data Repository 280 by generating database query commands specifying shelter resource data tables and relevant selection criteria. The Predictive Analytics Engine 240 may retrieve SDOH data from the Data Repository 280 by generating database query commands specifying SDOH data tables and relevant selection criteria.

The Predictive Analytics Engine 240 may combine retrieved data into a combined dataset by joining retrieved healthcare data, shelter management data, and SDOH data based on common individual identifiers, creating unified records that span clinical, housing, and social service domains. In some embodiments, the Predictive Analytics Engine 240 may analyze the combined dataset using a predictive analytics algorithm by applying statistical models, machine learning algorithms, or rule-based scoring systems to unified records. The predictive analytics algorithm may identify patterns in the combined dataset that correlate with adverse outcomes by evaluating combinations of clinical indicators, shelter utilization patterns, and SDOH factors that have historically preceded negative events.

The Predictive Analytics Engine 240 may generate a risk assessment by calculating risk scores for each individual based on identified patterns, comparing calculated risk scores to defined threshold values, and classifying individuals as at-risk when calculated risk scores exceed threshold values. In some embodiments, the risk assessment generated by the Predictive Analytics Engine 240 may identify at least one of a mental health crisis risk or a hospital readmission risk. The Predictive Analytics Engine 240 may identify mental health crisis risk by evaluating combinations of mental health diagnosis data, shelter stay patterns, medication adherence indicators, and recent service utilization that correlate with historical mental health crisis events. The Predictive Analytics Engine 240 may identify hospital readmission risk by evaluating combinations of medical history, recent hospitalization data, housing instability indicators, and social support factors that correlate with historical readmission events.

In some embodiments, the Predictive Analytics Engine 240 may be further configured to generate an intervention recommendation based on the risk assessment. The intervention recommendation may comprise at least one of a caseworker outreach notification, a mental health service referral, or a healthcare provider alert. The Predictive Analytics Engine 240 may generate caseworker outreach notifications by identifying at-risk individuals who would benefit from direct caseworker contact, formatting notification messages containing relevant individual information and recommended outreach actions, and transmitting formatted notifications to the Communication and User Interface Module 250 for delivery to appropriate caseworkers. The Predictive Analytics Engine 240 may generate mental health service referrals by identifying at-risk individuals with elevated mental health crisis risk, formatting referral messages containing relevant clinical information and recommended service providers, and transmitting formatted referrals to appropriate mental health service coordinators. The Predictive Analytics Engine 240 may generate healthcare provider alerts by identifying at-risk individuals with elevated hospital readmission risk, formatting alert messages containing relevant medical information and recommended preventive interventions, and transmitting formatted alerts to the Communication and User Interface Module 250 for delivery to appropriate healthcare providers.

In some embodiments, the Predictive Analytics Engine 240 or the Communication and User Interface Module 250 may facilitate a closed-loop referral workflow. The care coordination platform may receive a referral submission from a healthcare provider, shelter staff member, or caseworker identifying an individual and a requested service such as shelter placement, behavioral health services, or social service support. The care coordination platform may automatically match the referral to one or more receiving facilities or service providers based on resource availability data maintained by the Shelter Management Module 220, individual clinical needs identified in healthcare data, SDOH factors, and eligibility criteria configured in recommendation rules stored in the Data Repository 280. The matched receiving facility or service provider may receive a referral notification via the Communication and User Interface Module 250 and may submit an acceptance or denial response through the user interface. The care coordination platform may update a referral status record in the Data Repository 280 to reflect the acceptance or denial response, notify the originating user of the response, and when a referral is accepted, track the referred service through to completion. Upon completion of the referred service, the care coordination platform may log a referral outcome record documenting service delivery, individual status changes, and any follow-up actions required. The Audit Module 260 may generate audit records documenting each stage of the referral workflow including submission, matching, acceptance or denial, service delivery, and outcome logging.

In some embodiments, the Communication and User Interface Module 250 may be configured to provide access to the care coordination platform for a plurality of user types comprising at least healthcare providers, shelter staff, and caseworkers. The Communication and User Interface Module 250 may generate user interfaces that are transmitted to user computing devices 145 via the network 190 for display on device screens. In some embodiments, the Communication and User Interface Module 250 may implement authentication mechanisms by receiving user credentials from user computing devices 145, validating received credentials against stored credential records in the Data Repository 280, and granting or denying access based on validation results.

The Communication and User Interface Module 250 may provide role-based access by determining user type based on authenticated user credentials, retrieving role-specific interface configurations from the Data Repository 280, and generating interfaces that display information and functions appropriate to the determined user type. Healthcare providers may receive interfaces displaying clinical data alongside shelter status and SDOH metrics. Shelter staff may receive interfaces displaying bed availability and intake information alongside relevant health information for residents. Caseworkers may receive interfaces displaying comprehensive profiles spanning healthcare, shelter, and social service domains.

In some embodiments, the Communication and User Interface Module 250 may be configured to display a customizable dashboard presenting the healthcare data, the shelter management data, and the SDOH data. The Communication and User Interface Module 250 may generate customizable dashboards by retrieving dashboard configuration preferences for authenticated users, querying the Data Repository 280 via the Database Engine 270 for data elements specified in configuration preferences, formatting retrieved data elements according to layout specifications, and transmitting formatted dashboard displays to user computing devices 145.

In some embodiments, the Communication and User Interface Module 250 may be further configured to provide mobile access to the care coordination platform via a mobile computing device. The Communication and User Interface Module 250 may generate mobile-optimized interfaces by detecting device characteristics from connection requests, selecting interface templates appropriate for detected device characteristics, and formatting interface elements for display on mobile device screens. This mobile access may enable caseworkers to collect SDOH data during field visits, shelter staff to update resource availability from any location within facilities, and healthcare providers to access care coordination information during patient encounters.

In some embodiments, the Communication and User Interface Module 250 may be further configured to support offline data collection and synchronization via mobile computing devices. The Communication and User Interface Module 250 may enable mobile computing devices to cache data collection forms, individual records, and interface elements in local device storage when network connectivity is unavailable. Caseworkers operating in field environments where network access is intermittent or unavailable may enter SDOH data, record service delivery notes, and update individual records using locally cached interfaces. The Communication and User Interface Module 250 may implement a synchronization queue that stores data entries made during offline periods in local device storage, detects when network connectivity is restored, and transmits queued data entries to the computing system 100 via the network 190. The Database Engine 270 may process received queued entries by applying conflict resolution logic that compares timestamps of offline entries with timestamps of any intervening server-side modifications, retaining the most recent values or flagging conflicts for manual review when concurrent modifications are detected. Upon successful synchronization, the Communication and User Interface Module 250 may update locally cached data on the mobile computing device to reflect current server-side records, ensuring data consistency between mobile devices and the Data Repository 280. This offline synchronization capability may extend the operational reach of the care coordination platform to locations where persistent network connectivity cannot be guaranteed, such as outdoor encampments, rural service areas, or facilities with limited wireless infrastructure.

In some embodiments, the Audit Module 260 may be configured to generate a record documenting data access and data modifications by each of the plurality of user types. The Audit Module 260 may receive logging requests from other components of the application program 200 as those components execute their respective functions. In some embodiments, the Audit Module 260 may generate timestamped records by capturing current system time when logging requests are received, associating captured timestamps with logged actions, and storing timestamped records in the Data Repository 280 via the Database Engine 270.

The Audit Module 260 may document data access by recording user identifiers, timestamps, data elements accessed, and access purposes for each data retrieval operation performed by authenticated users. The Audit Module 260 may document data modifications by recording user identifiers, timestamps, data elements modified, previous values, new values, and modification purposes for each data update operation performed by authenticated users. In some embodiments, the Audit Module 260 may implement tamper-evident logging mechanisms by calculating hash values for stored audit records, storing calculated hash values alongside corresponding records, and enabling subsequent verification that stored records have not been modified after initial storage. This comprehensive documentation may support regulatory compliance requirements, quality improvement initiatives, and accountability for data handling across organizational boundaries.

In some embodiments, the application program 200 may further comprise a Behavioral Health Scheduling Module configured to coordinate scheduling of behavioral health services for individuals served by the care coordination platform. The Behavioral Health Scheduling Module may retrieve behavioral health provider availability from provider scheduling systems via the network 190, maintain a directory of behavioral health service providers including provider specialties, accepted insurance types, available appointment types, and geographic service areas, and match individuals with appropriate providers based on clinical needs identified in healthcare data stored in the Data Repository 280. The Behavioral Health Scheduling Module may generate appointment requests by identifying individuals with mental health diagnoses, substance use disorders, or behavioral health service needs documented in healthcare data or identified through risk assessments generated by the Predictive Analytics Engine 240, querying the provider directory for providers matching identified needs, retrieving available appointment slots from matching providers, and presenting available options to caseworkers or healthcare providers via the Communication and User Interface Module 250. When an appointment is confirmed, the Behavioral Health Scheduling Module may store scheduling records in the Data Repository 280 via the Database Engine 270, transmit appointment details to the individual's record in external EHR systems 285 via the Electronic Health Record Interface Module 210, and generate appointment reminder notifications for delivery to individuals or assigned caseworkers via the Communication and User Interface Module 250. The Behavioral Health Scheduling Module may track appointment attendance by receiving status updates indicating whether scheduled appointments were completed, cancelled, or missed, and may communicate attendance data to the Predictive Analytics Engine 240 for incorporation into risk assessment calculations where missed behavioral health appointments may indicate elevated risk levels.

In some embodiments, the Database Engine 270 may be configured to manage data storage and retrieval operations for the Data Repository 280. The Database Engine 270 may execute database queries submitted by other components of the application program 200 by parsing received query commands, optimizing query execution plans, executing optimized queries against stored data, and returning query results to requesting components. The Database Engine 270 may maintain indexes that enable efficient data retrieval by creating index structures based on frequently queried data fields, updating index structures when underlying data changes, and utilizing index structures to accelerate query execution.

The Database Engine 270 may ensure data integrity by enforcing constraints on stored data, validating data values before storage operations, and rejecting storage operations that would violate defined constraints. In some embodiments, the Database Engine 270 may support concurrent access by multiple components by implementing locking mechanisms that prevent conflicting operations, managing transaction boundaries that ensure atomic execution of related operations, and resolving conflicts when concurrent operations affect the same data elements.

In some embodiments, the Data Repository 280 may store structured data used by the application program 200. The Data Repository 280 may contain healthcare data including medical history records, mental health diagnosis data, treatment plans, and clinical encounter records. The Data Repository 280 may contain shelter management data including bed availability records, intake status records, food service availability records, and shelter utilization history. The Data Repository 280 may contain SDOH data including housing status records, employment status records, food access metrics, transportation access records, income level records, and education status records. The Data Repository 280 may contain risk assessment records including calculated risk scores, risk classifications, and intervention recommendations. The Data Repository 280 may contain audit logs including timestamped records of data access and data modifications. In some embodiments, the Data Repository 280 may be implemented as a relational database, a NoSQL database, or a distributed storage system depending on system requirements and data volumes.

In some embodiments, the server hosting the application program 200 may comprise a cloud-based server configured to synchronize data across a plurality of shelter facilities. The cloud-based server may maintain the Data Repository 280 in centralized cloud storage, receive data updates from shelter facility systems 290 at multiple locations via the network 190, propagate received updates to the centralized Data Repository 280, and enable user computing devices 145 at any connected location to access synchronized data. This cloud-based architecture may enable organizations operating multiple shelter locations, healthcare facilities, or service sites to maintain unified records without requiring manual data transfer between locations.

In some embodiments, the network 190 may be a public or private data network, such as the Internet or a corporate intranet, enabling communication between the computing system 100, user computing devices 145, external EHR systems 285, and shelter facility systems 290. The network 190 may utilize standard communication protocols such as TCP/IP, HTTPS, or other network protocols to facilitate data transmission between connected systems. In some embodiments, the network 190 may support both wired and wireless connections, enabling mobile devices to access the care coordination platform from field locations and enabling shelter facility systems 290 to transmit data from distributed shelter sites.

User computing devices 145 may be mobile devices such as smartphones or tablets, portable computing devices such as laptops, or fixed workstations such as desktop computers. In some embodiments, user computing devices 145 may execute web browsers or dedicated mobile applications that interface with the Communication and User Interface Module 250 to display care coordination interfaces, risk assessments, and intervention recommendations. Users may interact with user computing devices 145 to access healthcare data, shelter management data, and SDOH data, view risk assessments for individuals, and respond to intervention recommendations. The user computing devices 145 may communicate with the computing system 100 via the network 190 using encrypted connections to maintain data security during transmission of healthcare and personal information.

External EHR systems 285 may be commercially available electronic health record platforms such as Epic, Cerner, or other healthcare information systems that maintain clinical records for patient populations. In some embodiments, external EHR systems 285 may expose FHIR-based APIs that enable standardized data exchange with external systems. The Electronic Health Record Interface Module 210 may establish connections with external EHR systems 285 via the network 190 using these standardized APIs to retrieve clinical data and transmit updated data. The integration between the Electronic Health Record Interface Module 210 and external EHR systems 285 may enable the care coordination platform to access current clinical information without requiring manual data entry or duplicate record maintenance in multiple systems.

Shelter facility systems 290 may be shelter management software platforms, occupancy tracking databases, or intake management systems operated by homeless shelters, transitional housing facilities, or other shelter providers. In some embodiments, shelter facility systems 290 may maintain records of bed availability, intake queues, resident information, and service offerings. The Shelter Management Module 220 may establish connections with shelter facility systems 290 via the network 190 using APIs, database connections, or file transfer protocols to retrieve current shelter resource data. The integration between the Shelter Management Module 220 and shelter facility systems 290 may enable the care coordination platform to access real-time shelter information without requiring shelter staff to manually report resource availability through separate communication channels.

In some embodiments, the computing system 100 may communicate via the network 190 with one or more government healthcare program systems including systems associated with Medicaid, Medicare, or other government-administered healthcare coverage programs. The Electronic Health Record Interface Module 210 or a dedicated interoperability interface may exchange data with government healthcare program systems to retrieve eligibility indicators confirming whether individuals are enrolled in or eligible for government healthcare coverage, coverage status data indicating active enrollment periods and covered service categories, prior authorization status data indicating whether specific services or referrals have received required approvals, and coordination of benefits data indicating relationships between government coverage and other insurance sources. The interoperability interface may transmit data to government healthcare program systems comprising service delivery records documenting care coordination activities, referral status data documenting referral outcomes and service utilization, and reporting data supporting program compliance and outcome measurement requirements. The data exchange with government healthcare program systems may be implemented via standardized interoperability protocols including FHIR-based APIs, X12 Electronic Data Interchange (EDI) transaction sets for eligibility inquiries, or program-specific data exchange interfaces defined by state Medicaid information systems or federal Medicare systems. The retrieved eligibility and coverage data may be stored in the Data Repository 280 via the Database Engine 270 and associated with individual records, enabling healthcare providers, shelter staff, and caseworkers to view coverage status alongside clinical data, shelter data, and SDOH data through the Communication and User Interface Module 250. This government program interoperability may enable the care coordination platform to verify coverage eligibility before initiating referrals, identify individuals who may qualify for but are not currently enrolled in government healthcare programs, and generate reporting data required by government program administrators without requiring manual data extraction and reformatting.

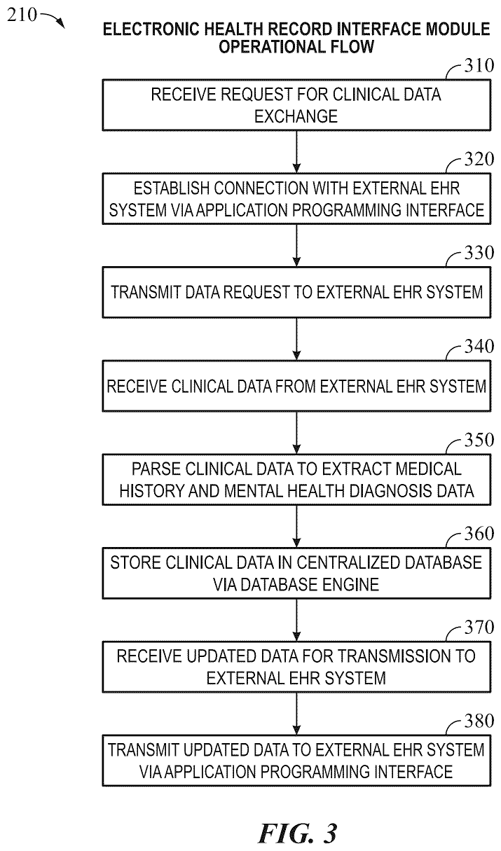

FIG. 3 is a flow diagram illustrating an exemplary operational sequence of the Electronic Health Record Interface Module 210 in accordance with certain embodiments. The sequence shown may be implemented as computer-executable instructions within the application program 200 operating on computing system 100 of FIG. 2, with specific operations performed by the modules depicted therein.

At step 310, a request for clinical data exchange is received. This operation may be performed by the Electronic Health Record Interface Module 210, which receives data exchange requests from other components of the application program 200 or from user computing devices 145 via the Communication and User Interface Module 250. In some embodiments, the request for clinical data exchange may specify one or more individuals for whom clinical data is requested, data types to be retrieved such as medical history or mental health diagnosis data, and external EHR systems 285 from which data should be retrieved. The Electronic Health Record Interface Module 210 may parse received requests to extract request parameters including individual identifiers, requested data types, and target external EHR systems 285. In some embodiments, the request for clinical data exchange may be triggered automatically by other components of the application program 200, such as when the Predictive Analytics Engine 240 requires current clinical data to generate risk assessments, or may be triggered manually by authenticated users seeking to view clinical information for specific individuals.

At step 320, a connection is established with the external EHR system via an application programming interface (API). This operation is performed by the Electronic Health Record Interface Module 210, which initiates communication sessions with external EHR systems 285 via the network 190. In some embodiments, the Electronic Health Record Interface Module 210 may establish the connection using a Fast Healthcare Interoperability Resources (FHIR)-based API configured to enable bidirectional data exchange between the care coordination platform and the at least one external EHR system 285. The Electronic Health Record Interface Module 210 may establish the connection by resolving network addresses for target external EHR systems 285, initiating TCP/IP connections to resolved addresses, performing TLS handshake procedures to establish encrypted communication channels, and authenticating with external EHR systems 285 using OAuth 2.0 tokens, API keys, or other authentication credentials.

In some embodiments, the Electronic Health Record Interface Module 210 may maintain a registry of external EHR systems 285 with which connections may be established, including network addresses, authentication credentials, supported API versions, and available data resources for each registered system. The Electronic Health Record Interface Module 210 may retrieve connection parameters from this registry based on target system identifiers specified in the data exchange request received at step 310. In some embodiments, the Electronic Health Record Interface Module 210 may implement connection pooling by maintaining persistent connections with frequently accessed external EHR systems 285 to reduce connection establishment latency for subsequent requests.

At step 330, a data request is transmitted to the external EHR system. This operation is performed by the Electronic Health Record Interface Module 210, which formats and transmits data requests according to FHIR specifications. In some embodiments, the Electronic Health Record Interface Module 210 may construct FHIR search requests by specifying resource types to be retrieved such as Patient, Condition, Observation, or DiagnosticReport resources, search parameters that identify specific individuals or filter results based on clinical criteria, and data elements to be included in response messages. The Electronic Health Record Interface Module 210 may format constructed requests as HTTP GET or POST messages according to FHIR RESTful API conventions and transmit formatted messages to external EHR systems 285 via the established connection.

In some embodiments, the Electronic Health Record Interface Module 210 may transmit requests for multiple resource types in a single batch request by constructing FHIR Bundle resources containing multiple request entries. The Electronic Health Record Interface Module 210 may include request headers specifying accepted response formats such as JSON or XML, preferred response content such as minimal or full resource representations, and pagination parameters for large result sets. In some embodiments, the data request may specify clinical data comprising at least one of medical history or mental health diagnosis data by including search parameters that filter results to Condition resources with relevant clinical codes or DiagnosticReport resources containing mental health assessments.

At step 340, clinical data is received from the external EHR system. This operation is performed by the Electronic Health Record Interface Module 210, which receives response messages from external EHR systems 285 via the network 190. In some embodiments, the Electronic Health Record Interface Module 210 may receive HTTP response messages containing FHIR Bundle resources with requested clinical data, parse response headers to determine response status and content characteristics, and extract response body content for further processing. The Electronic Health Record Interface Module 210 may validate received responses by verifying response status codes indicate successful completion, confirming response content types match expected formats, and checking response content against FHIR resource schemas to ensure structural validity.

In some embodiments, the Electronic Health Record Interface Module 210 may handle paginated responses by detecting pagination links in received Bundle resources, transmitting follow-up requests to retrieve additional result pages, and aggregating results from multiple response pages into unified result sets. The Electronic Health Record Interface Module 210 may also handle error responses by detecting error status codes, parsing error details from response content, logging error information for diagnostic purposes, and generating appropriate error notifications for requesting components or users. In some embodiments, the Electronic Health Record Interface Module 210 may be configured to exchange the healthcare data with the at least one external EHR system 285 in real-time by processing responses immediately upon receipt and initiating subsequent processing steps without queuing delays.

At step 350, clinical data is parsed to extract medical history and mental health diagnosis data. This operation is performed by the Electronic Health Record Interface Module 210, which processes received FHIR resources to extract relevant clinical information. In some embodiments, the Electronic Health Record Interface Module 210 may parse received clinical data by traversing FHIR resource structures, identifying data elements within resource hierarchies, and extracting values from identified elements. The Electronic Health Record Interface Module 210 may extract medical history data by identifying Condition resources representing past and current diagnoses, extracting diagnosis codes from code elements using standardized coding systems such as ICD-10 or SNOMED CT, extracting diagnosis descriptions from display elements, and extracting temporal information from onset and abatement elements.

The Electronic Health Record Interface Module 210 may extract mental health diagnosis data by identifying Condition resources with diagnosis codes falling within mental health categories, extracting specific mental health diagnosis codes and descriptions, and extracting associated clinical details such as severity indicators, treatment status, and managing providers. In some embodiments, the Electronic Health Record Interface Module 210 may also extract data from related resources such as Encounter resources documenting clinical visits, Observation resources containing clinical measurements, and MedicationRequest resources indicating prescribed treatments. The Electronic Health Record Interface Module 210 may convert extracted values into internal data formats by mapping FHIR data types to internal data types, transforming coded values according to internal coding conventions, and structuring extracted data into internal record formats compatible with the Data Repository 280.

At step 360, clinical data is stored in the centralized database via the database engine. This operation is performed by the Electronic Health Record Interface Module 210 in coordination with the Database Engine 270. In some embodiments, the Electronic Health Record Interface Module 210 may prepare clinical data for storage by validating extracted data against defined data quality rules, associating extracted data with individual records based on matched individual identifiers, and formatting validated data into database record structures. The Electronic Health Record Interface Module 210 may transmit prepared data to the Database Engine 270 by generating database insert or update commands, specifying target tables and data fields, and including formatted data values.

The Database Engine 270 may execute received commands by parsing command syntax, validating data values against table constraints, and performing insert or update operations on the Data Repository 280. In some embodiments, the Database Engine 270 may implement transaction management by grouping related insert and update operations into atomic transactions, committing transactions when all operations complete successfully, and rolling back transactions when any operation fails. The Database Engine 270 may return confirmation responses to the Electronic Health Record Interface Module 210 indicating successful storage or providing error details when storage operations fail. In some embodiments, the Electronic Health Record Interface Module 210 may update existing clinical data records when more recent data is received from external EHR systems 285, maintaining current clinical information in the Data Repository 280 without creating duplicate records.

At step 370, updated data is received for transmission to the external EHR system. This operation is performed by the Electronic Health Record Interface Module 210, which receives data that has been created or modified within the care coordination platform and requires synchronization with external EHR systems 285. In some embodiments, the Electronic Health Record Interface Module 210 may receive updated data from other components of the application program 200, such as clinical observations recorded by healthcare providers via the Communication and User Interface Module 250, care coordination notes documenting service delivery, or updated assessment information generated by caseworkers. The Electronic Health Record Interface Module 210 may also receive updated data from the Data Repository 280 via the Database Engine 270 when data modifications trigger synchronization requirements.

In some embodiments, the Electronic Health Record Interface Module 210 may identify data requiring synchronization by maintaining synchronization status flags for data records, detecting records flagged for synchronization, and retrieving flagged records for transmission processing. The Electronic Health Record Interface Module 210 may validate updated data before transmission by verifying data completeness, confirming data formats comply with FHIR specifications, and checking data values against defined validation rules. In some embodiments, the Electronic Health Record Interface Module 210 may queue updated data for batch transmission when real-time synchronization is not required, aggregating multiple updates for efficient transmission during scheduled synchronization windows.

At step 380, updated data is transmitted to the external EHR system via the application programming interface. This operation is performed by the Electronic Health Record Interface Module 210, which formats and transmits updated data to external EHR systems 285. In some embodiments, the Electronic Health Record Interface Module 210 may format updated data by constructing FHIR resources representing updated clinical information, populating resource elements with data values from internal records, and encoding constructed resources in JSON or XML formats according to external system preferences. The Electronic Health Record Interface Module 210 may transmit formatted data by establishing or reusing connections with target external EHR systems 285, constructing HTTP POST or PUT requests containing formatted resources, and transmitting constructed requests via the network 190.

In some embodiments, the Electronic Health Record Interface Module 210 may transmit updates for multiple resources in batch requests by constructing FHIR Bundle resources containing multiple update entries and transmitting Bundle resources as single HTTP requests. The Electronic Health Record Interface Module 210 may process transmission responses by receiving HTTP response messages from external EHR systems 285, parsing response content to determine update outcomes for each transmitted resource, and updating synchronization status flags in the Data Repository 280 to reflect successful transmissions. In some embodiments, the Electronic Health Record Interface Module 210 may implement retry logic for failed transmissions by detecting transmission failures from error responses or network timeouts, queuing failed transmissions for retry attempts, and applying exponential backoff delays between retry attempts to avoid overwhelming external systems.

In some embodiments, the operations of steps 310 through 380 occur automatically upon receipt of data exchange requests or upon detection of data requiring synchronization, while in other embodiments they may be initiated on-demand by authorized users through control interfaces connected to the computing system 100. The automated execution of these steps enables real-time clinical data exchange that maintains synchronized records between the care coordination platform and external EHR systems 285 without requiring manual data entry or duplicate record maintenance. The bidirectional data exchange enabled by the Electronic Health Record Interface Module 210 ensures that clinical information flows seamlessly between the care coordination platform and existing healthcare infrastructure, enabling healthcare providers, shelter staff, and caseworkers to access current clinical data alongside shelter management data and social determinants of health data through a unified platform.

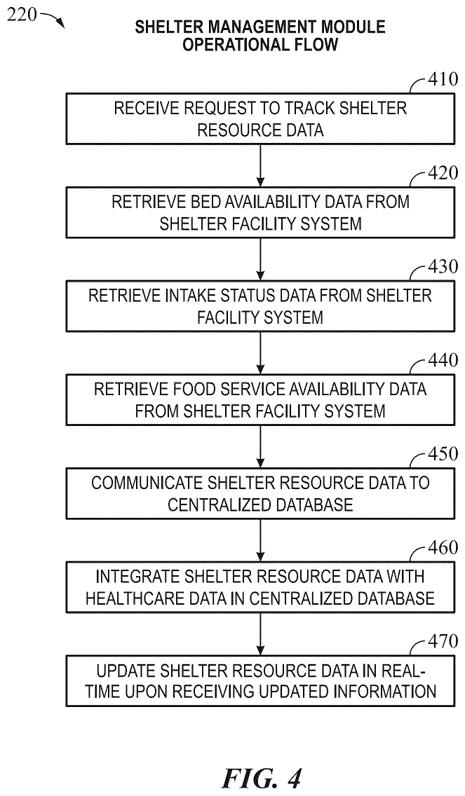

FIG. 4 is a flow diagram illustrating an exemplary operational sequence of the Shelter Management Module 220 in accordance with certain embodiments. The sequence shown may be implemented as computer-executable instructions within the application program 200 operating on computing system 100 of FIG. 2, with specific operations performed by the modules depicted therein.

At step 410, a request to track shelter resource data is received. This operation may be performed by the Shelter Management Module 220, which receives tracking requests from other components of the application program 200 or from external triggers indicating that shelter resource data should be retrieved. In some embodiments, the request to track shelter resource data may originate from the Predictive Analytics Engine 240 when current shelter information is required for risk assessment generation, from the Communication and User Interface Module 250 when users request to view shelter availability for specific facilities, or from scheduled tasks configured to retrieve shelter data at defined intervals. The Shelter Management Module 220 may parse received requests to extract request parameters including shelter facility identifiers specifying which shelter facility systems 290 should be queried, resource types to be tracked such as bed availability, intake status, or food service availability, and time ranges for historical data retrieval when applicable.

In some embodiments, the Shelter Management Module 220 may receive requests to track shelter resource data through multiple channels. The Shelter Management Module 220 may receive synchronous requests via internal method calls from other modules of the application program 200, requiring immediate response with current shelter data. The Shelter Management Module 220 may receive asynchronous requests via message queues, enabling decoupled processing where shelter data retrieval occurs independently of requesting components. The Shelter Management Module 220 may also receive event-driven requests triggered by webhooks or push notifications from shelter facility systems 290 indicating that resource availability has changed and updated data should be retrieved.

At step 420, bed availability data is retrieved from the shelter facility system. This operation is performed by the Shelter Management Module 220, which establishes communication connections with shelter facility systems 290 via the network 190 and retrieves current bed availability information. In some embodiments, the Shelter Management Module 220 may establish connections with shelter facility systems 290 by resolving network addresses for target systems from a configuration registry, initiating TCP/IP connections to resolved addresses, performing authentication procedures using API keys, username/password credentials, or certificate-based authentication, and establishing secure communication channels using TLS encryption.

The Shelter Management Module 220 may retrieve bed availability data by constructing API requests specifying bed availability resources, transmitting constructed requests to shelter facility systems 290 via established connections, and receiving response messages containing requested bed availability information. In some embodiments, the Shelter Management Module 220 may construct requests according to API specifications defined by shelter facility systems 290, which may include RESTful API conventions, SOAP web service protocols, or proprietary data exchange formats. The Shelter Management Module 220 may specify query parameters in constructed requests to filter results by shelter location, bed type such as individual beds, family units, or medical beds, gender restrictions, age restrictions, or accessibility requirements.

In some embodiments, the bed availability data retrieved by the Shelter Management Module 220 may comprise total bed capacity indicating the maximum number of beds available at each shelter facility, current occupancy count indicating the number of beds currently in use, available bed count calculated as the difference between total capacity and current occupancy, bed reservation count indicating beds reserved for incoming individuals, and projected availability indicating expected bed availability based on scheduled departures. The Shelter Management Module 220 may parse received response messages by extracting data values from response structures, converting extracted values from external data formats to internal data formats, and validating extracted values against expected data types and value ranges to ensure data quality.

At step 430, intake status data is retrieved from the shelter facility system. This operation is performed by the Shelter Management Module 220, which queries shelter facility systems 290 for current intake processing information. In some embodiments, the Shelter Management Module 220 may retrieve intake status data by constructing API requests specifying intake status resources, transmitting constructed requests to shelter facility systems 290 via established connections or by reusing connections established during step 420, and receiving response messages containing requested intake status information. The Shelter Management Module 220 may construct intake status requests by specifying resource endpoints corresponding to intake queues, waitlists, or intake processing workflows maintained by shelter facility systems 290.

In some embodiments, the intake status data retrieved by the Shelter Management Module 220 may comprise intake queue length indicating the number of individuals currently waiting for intake processing, average intake processing time calculated from historical intake records, current intake capacity indicating the number of intake slots available for new arrivals, intake eligibility criteria specifying requirements that individuals must satisfy for shelter admission, and intake hours indicating time periods during which intake processing is available. The Shelter Management Module 220 may parse received intake status data by traversing response data structures, identifying elements containing intake-related information, extracting values from identified elements, and converting extracted values to internal data formats compatible with the Data Repository 280.

In some embodiments, the Shelter Management Module 220 may retrieve intake status data that includes individual-level intake records when authorized by data sharing agreements between the care coordination platform and shelter facility systems 290. Individual-level intake records may include individual identifiers enabling linkage with healthcare data and social determinants of health data stored in the Data Repository 280, intake timestamps indicating when individuals were admitted to shelters, intake source information indicating how individuals were referred to shelters, and intake assessment results documenting needs identified during intake processing. The Shelter Management Module 220 may apply data privacy protections when processing individual-level intake records by filtering records to include only individuals who have consented to data sharing, masking sensitive data elements according to configured privacy rules, and logging data access events for audit purposes via the Audit Module 260.

At step 440, food service availability data is retrieved from the shelter facility system. This operation is performed by the Shelter Management Module 220, which queries shelter facility systems 290 for current food service information. In some embodiments, the Shelter Management Module 220 may be further configured to track food service availability for the at least one shelter facility by constructing API requests specifying food service resources, transmitting constructed requests to shelter facility systems 290, and receiving response messages containing requested food service information. The Shelter Management Module 220 may construct food service requests by specifying resource endpoints corresponding to meal schedules, food inventory, or nutrition services maintained by shelter facility systems 290.

In some embodiments, the food service availability data retrieved by the Shelter Management Module 220 may comprise meal schedules indicating times when breakfast, lunch, dinner, and snacks are served, meal capacity indicating the maximum number of individuals that can be served during each meal period, current meal availability indicating remaining capacity for upcoming meal periods, dietary accommodation options indicating availability of meals meeting specific dietary requirements such as vegetarian, halal, kosher, gluten-free, or allergen-free options, and food pantry availability indicating whether food items are available for individuals to take for consumption outside scheduled meal times. The Shelter Management Module 220 may parse received food service data by identifying meal-related elements within response structures, extracting schedule information, capacity values, and accommodation details, and converting extracted values to internal data formats.

In some embodiments, the Shelter Management Module 220 may retrieve food service data from multiple sources within shelter facility systems 290. The Shelter Management Module 220 may query kitchen management systems for meal preparation schedules and capacity information. The Shelter Management Module 220 may query inventory management systems for food stock levels and expected delivery dates. The Shelter Management Module 220 may query nutrition services databases for dietary accommodation capabilities and special diet request processing status. The Shelter Management Module 220 may aggregate data retrieved from multiple sources into unified food service availability records that provide comprehensive views of nutrition resources available at each shelter facility.

At step 450, shelter resource data is communicated to the centralized database. This operation is performed by the Shelter Management Module 220, which transmits retrieved shelter resource data to the Database Engine 270 for storage in the Data Repository 280. In some embodiments, the Shelter Management Module 220 may communicate the shelter resource data to the centralized database by aggregating bed availability data retrieved at step 420, intake status data retrieved at step 430, and food service availability data retrieved at step 440 into unified shelter resource records. The Shelter Management Module 220 may format aggregated data into database record structures by mapping retrieved data elements to corresponding database fields, converting data types as required by database schema definitions, and generating unique record identifiers for new shelter resource records.

The Shelter Management Module 220 may transmit formatted shelter resource data to the Database Engine 270 by generating structured query language (SQL) insert statements for new records or update statements for existing records, specifying target database tables and field mappings, including formatted data values as statement parameters, and transmitting generated statements to the Database Engine 270 via internal method calls or database connection interfaces. In some embodiments, the Shelter Management Module 220 may utilize object-relational mapping (ORM) frameworks to translate between internal data objects and database records, enabling the Shelter Management Module 220 to interact with the Data Repository 280 using object-oriented programming conventions rather than direct SQL statement construction.

In some embodiments, the Database Engine 270 may execute received insert or update statements by parsing statement syntax, validating data values against table constraints and foreign key relationships, acquiring database locks to prevent concurrent modification conflicts, performing insert or update operations on target tables within the Data Repository 280, and releasing acquired locks upon operation completion. The Database Engine 270 may return execution results to the Shelter Management Module 220 indicating successful storage, the number of records affected, or error details when storage operations fail. The Shelter Management Module 220 may process returned results by logging successful storage events, updating internal tracking structures to reflect current synchronization status, and initiating error handling procedures when storage failures occur.

At step 460, shelter resource data is integrated with healthcare data in the centralized database. This operation is performed by the Shelter Management Module 220 in coordination with the Database Engine 270, which establishes relationships between shelter resource records and healthcare records stored in the Data Repository 280. In some embodiments, the Shelter Management Module 220 may integrate shelter resource data with healthcare data by identifying common individual identifiers that link shelter records to healthcare records, creating association records that establish relationships between shelter utilization data and clinical data for the same individuals, and updating individual profile records to include references to associated shelter resource data.

The Shelter Management Module 220 may perform integration by generating SQL join queries that combine shelter resource tables with healthcare data tables based on matching individual identifiers, executing generated queries via the Database Engine 270 to retrieve linked record sets, and storing query results in integrated view tables or materialized views that enable efficient access to combined data. In some embodiments, the Shelter Management Module 220 may create database foreign key relationships between shelter resource tables and healthcare data tables by defining referential integrity constraints that enforce valid relationships between linked records. The Database Engine 270 may enforce these constraints by validating that referenced records exist before allowing insert operations and preventing deletion of records that are referenced by other tables.

In some embodiments, the integration performed at step 460 may enable the Predictive Analytics Engine 240 to analyze combined datasets comprising healthcare data and shelter management data without requiring separate data retrieval and joining operations. The Shelter Management Module 220 may create indexed views that pre-compute commonly accessed combinations of shelter and healthcare data, enabling rapid query execution when the Predictive Analytics Engine 240 retrieves data for risk assessment generation. The Shelter Management Module 220 may also create event triggers that automatically update integrated views when underlying shelter resource data or healthcare data changes, ensuring that integrated data remains current without requiring explicit refresh operations.

In some embodiments, the integration may enable healthcare providers, shelter staff, and caseworkers to access shelter status information alongside clinical records through the Communication and User Interface Module 250. The integrated data may enable healthcare providers to view current shelter status for patients being discharged, facilitating coordination between discharge planning and shelter placement. The integrated data may enable shelter staff to view relevant health information for residents, enabling appropriate accommodation of medical needs. The integrated data may enable caseworkers to view comprehensive profiles spanning clinical and housing domains, supporting holistic assessment of individual needs and service coordination.

At step 470, shelter resource data is updated in real-time upon receiving updated information. This operation is performed by the Shelter Management Module 220, which monitors for changes in shelter resource availability and updates stored data accordingly. In some embodiments, the Shelter Management Module 220 may be configured to track the shelter resource data in real-time by implementing multiple update detection mechanisms. The Shelter Management Module 220 may implement polling-based updates by scheduling periodic queries to shelter facility systems 290 at defined intervals such as every five minutes, every fifteen minutes, or every hour depending on configured update frequency requirements. The Shelter Management Module 220 may compare newly retrieved data with previously stored data to detect changes, and may update the Data Repository 280 only when changes are detected to minimize unnecessary database operations.

In some embodiments, the Shelter Management Module 220 may implement push-based updates by registering webhook endpoints with shelter facility systems 290 that support event notification capabilities. When shelter resource availability changes, shelter facility systems 290 may transmit HTTP POST requests to registered webhook endpoints containing updated resource data. The Shelter Management Module 220 may receive webhook notifications via the network 190, validate received notifications to confirm they originate from authorized shelter facility systems 290, parse notification content to extract updated resource data, and update corresponding records in the Data Repository 280 via the Database Engine 270. Push-based updates may enable more timely data synchronization than polling-based updates by eliminating latency between resource changes and update detection.

In some embodiments, the Shelter Management Module 220 may implement streaming-based updates by establishing persistent connections with shelter facility systems 290 using WebSocket protocols or server-sent events. Shelter facility systems 290 may transmit resource updates through established streaming connections as changes occur, enabling continuous real-time synchronization without repeated connection establishment overhead. The Shelter Management Module 220 may process received streaming updates by parsing update messages, validating update content, and applying updates to the Data Repository 280 in near real-time. The Shelter Management Module 220 may implement reconnection logic to re-establish streaming connections when network interruptions occur, ensuring continuous update reception despite transient connectivity issues.

In some embodiments, the Shelter Management Module 220 may implement change detection by maintaining hash values or version identifiers for stored shelter resource records. When updated data is received through any update mechanism, the Shelter Management Module 220 may calculate hash values for received data and compare calculated hashes with stored hashes to determine whether received data differs from previously stored data. When hash comparison indicates that data has changed, the Shelter Management Module 220 may update stored records and stored hash values. When hash comparison indicates that data has not changed, the Shelter Management Module 220 may skip database update operations to conserve processing resources. The Shelter Management Module 220 may also maintain timestamp fields indicating when each shelter resource record was last updated, enabling other components of the application program 200 to determine data currency when accessing shelter information.

In some embodiments, the real-time update capability may enable the care coordination platform to maintain current shelter resource information that reflects actual availability rather than potentially outdated information retrieved during previous synchronization cycles. The Shelter Management Module 220 may propagate update notifications to other components of the application program 200 when shelter resource data changes. The Shelter Management Module 220 may notify the Predictive Analytics Engine 240 when shelter utilization patterns change, potentially triggering re-evaluation of risk assessments for individuals whose shelter status has changed. The Shelter Management Module 220 may notify the Communication and User Interface Module 250 to refresh displayed shelter information for users currently viewing shelter availability data. The Shelter Management Module 220 may generate audit records via the Audit Module 260 documenting when shelter resource data was updated, what values changed, and what sources provided updated information.

In some embodiments, the operations of steps 410 through 470 occur automatically based on configured schedules, event triggers, or streaming connections, while in other embodiments they may be initiated on-demand by authorized users or other components of the application program 200. The automated execution of these steps enables continuous tracking of shelter resource availability across one or more shelter facilities without requiring manual data collection or entry by shelter staff or care coordinators. By communicating shelter resource data to the centralized database and integrating shelter resource data with healthcare data, the Shelter Management Module 220 enables the care coordination platform to provide unified access to shelter and clinical information that was previously maintained in separate, disconnected systems. The real-time update capability ensures that shelter resource data reflects current conditions, enabling healthcare providers, shelter staff, and caseworkers to make informed decisions based on accurate availability information when coordinating services for individuals requiring both healthcare and shelter support.

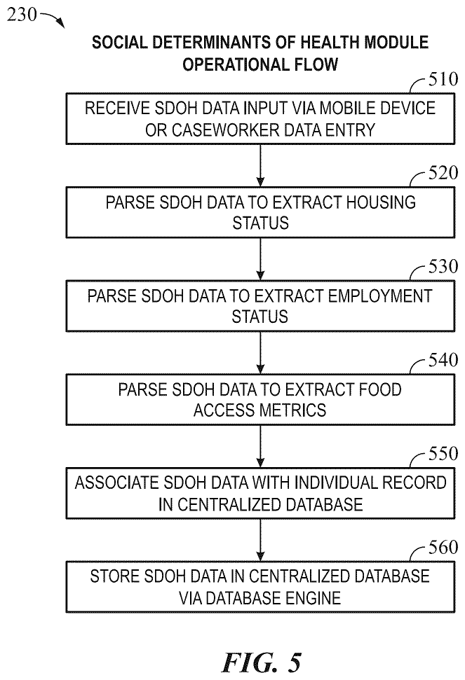

FIG. 5 is a flow diagram illustrating an exemplary operational sequence of the Social Determinants of Health Module 230 in accordance with certain embodiments. The sequence shown may be implemented as computer-executable instructions within the application program 200 operating on computing system 100 of FIG. 2, with specific operations performed by the modules depicted therein.

At step 510, social determinants of health (SDOH) data input is received via a mobile device or caseworker data entry. This operation may be performed by the Social Determinants of Health Module 230, which receives SDOH data from user computing devices 145 via the network 190. In some embodiments, the Social Determinants of Health Module 230 may be configured to receive the SDOH data via at least one of a mobile device input or a caseworker data entry. The Social Determinants of Health Module 230 may receive mobile device input by generating data collection forms that are transmitted to mobile computing devices via the Communication and User Interface Module 250, receiving completed form submissions via HTTP POST requests transmitted from mobile devices through the network 190, and parsing received request content to extract submitted form data.

In some embodiments, the Social Determinants of Health Module 230 may generate data collection forms by retrieving form templates from the Data Repository 280 via the Database Engine 270, populating template fields with individual-specific information when forms are generated for known individuals, and formatting populated templates for display on mobile device screens. The data collection forms may include input fields for housing status, employment status, food access metrics, and additional SDOH factors. The Social Determinants of Health Module 230 may configure form fields with appropriate input types such as dropdown selections for categorical data, numeric inputs for quantitative data, date pickers for temporal data, and text areas for narrative descriptions.

In some embodiments, the Social Determinants of Health Module 230 may receive caseworker data entry through web-based interfaces displayed on user computing devices 145 including desktop computers, laptop computers, or tablet devices. The Social Determinants of Health Module 230 may generate caseworker data entry interfaces by retrieving interface configurations from the Data Repository 280, rendering interface layouts according to retrieved configurations, and transmitting rendered interfaces to user computing devices 145 via the Communication and User Interface Module 250. Caseworkers may enter SDOH data by navigating to individual records, selecting data entry options, completing displayed forms, and submitting entered data for processing.

The Social Determinants of Health Module 230 may validate received SDOH data input by checking that required fields contain values, verifying that entered values conform to expected data types and formats, confirming that categorical values match defined option sets, and validating that numeric values fall within acceptable ranges. In some embodiments, the Social Determinants of Health Module 230 may return validation error messages to submitting users when validation failures are detected, identifying specific fields requiring correction and providing guidance on acceptable values. The Social Determinants of Health Module 230 may accept validated submissions for further processing and reject invalid submissions until corrections are made.

At step 520, SDOH data is parsed to extract housing status. This operation is performed by the Social Determinants of Health Module 230, which processes received SDOH data to identify and extract housing-related information. In some embodiments, the Social Determinants of Health Module 230 may parse SDOH data to extract housing status by identifying form fields or data elements designated as housing status indicators, extracting values from identified fields, and converting extracted values to standardized housing status categories. The Social Determinants of Health Module 230 may identify housing status fields by matching field identifiers against a housing field registry maintained in the Data Repository 280, enabling flexible configuration of which form fields contribute to housing status determination.

In some embodiments, the housing status extracted by the Social Determinants of Health Module 230 may comprise categorical classifications indicating current living situation. Housing status categories may include unsheltered indicating individuals living outdoors, in vehicles, or in locations not intended for human habitation; emergency shelter indicating individuals residing in homeless shelters or transitional housing facilities; transitional housing indicating individuals in temporary housing programs with defined duration limits; permanent supportive housing indicating individuals in long-term housing with integrated support services; and permanent housing indicating individuals in stable rental or owned housing without specialized support requirements.

The Social Determinants of Health Module 230 may extract additional housing-related data elements beyond categorical status. In some embodiments, the Social Determinants of Health Module 230 may extract housing duration indicating how long individuals have been in their current housing situation, housing history indicating previous housing situations and transitions, housing barriers indicating factors preventing individuals from obtaining or maintaining stable housing, and housing goals indicating individual preferences and targets for future housing situations. The Social Determinants of Health Module 230 may convert extracted housing data to internal data formats by mapping external category codes to internal category identifiers, standardizing date formats for temporal data, and normalizing text values for consistent storage and retrieval.

At step 530, SDOH data is parsed to extract employment status. This operation is performed by the Social Determinants of Health Module 230, which processes received SDOH data to identify and extract employment-related information. In some embodiments, the Social Determinants of Health Module 230 may parse SDOH data to extract employment status by identifying form fields or data elements designated as employment status indicators, extracting values from identified fields, and converting extracted values to standardized employment status categories. The Social Determinants of Health Module 230 may identify employment status fields by matching field identifiers against an employment field registry maintained in the Data Repository 280.

In some embodiments, the employment status extracted by the Social Determinants of Health Module 230 may comprise categorical classifications indicating current work situation. Employment status categories may include employed full-time indicating individuals working thirty-five or more hours per week in regular employment; employed part-time indicating individuals working fewer than thirty-five hours per week; self-employed indicating individuals operating their own businesses or working as independent contractors; unemployed seeking work indicating individuals without employment who are actively seeking employment opportunities; unemployed not seeking work indicating individuals without employment who are not currently seeking employment; and unable to work indicating individuals who cannot work due to disability, illness, or other barriers.

The Social Determinants of Health Module 230 may extract additional employment-related data elements beyond categorical status. In some embodiments, the Social Determinants of Health Module 230 may extract employment duration indicating how long individuals have been in their current employment status, employment history indicating previous employment positions and durations, income level indicating current earnings from employment or other sources, employment barriers indicating factors preventing individuals from obtaining or maintaining employment, and employment goals indicating individual career objectives and job search targets. The Social Determinants of Health Module 230 may convert extracted employment data to internal data formats by mapping external category codes to internal category identifiers and normalizing income values to standard currency units and time periods.

At step 540, SDOH data is parsed to extract food access metrics. This operation is performed by the Social Determinants of Health Module 230, which processes received SDOH data to identify and extract nutrition-related information. In some embodiments, the Social Determinants of Health Module 230 may parse SDOH data to extract food access metrics by identifying form fields or data elements designated as food access indicators, extracting values from identified fields, and converting extracted values to standardized food access measurements. The Social Determinants of Health Module 230 may identify food access fields by matching field identifiers against a food access field registry maintained in the Data Repository 280.

In some embodiments, the food access metrics extracted by the Social Determinants of Health Module 230 may comprise indicators of food security status and access to nutrition resources. Food access metrics may include food security classification indicating whether individuals have consistent access to adequate food, with categories such as high food security, marginal food security, low food security, and very low food security. Food access metrics may include meal frequency indicating the average number of meals consumed per day. Food access metrics may include food assistance enrollment indicating participation in programs such as the Supplemental Nutrition Assistance Program (SNAP), food pantry utilization, or shelter meal services.

The Social Determinants of Health Module 230 may extract additional food access data elements. In some embodiments, the Social Determinants of Health Module 230 may extract dietary restrictions indicating medical, religious, or personal dietary requirements that affect food access; food access barriers indicating factors such as transportation limitations, financial constraints, or geographic distance from food sources that limit access to adequate nutrition; and nutrition goals indicating individual objectives for improving food access or dietary habits. The Social Determinants of Health Module 230 may calculate composite food access scores by applying weighted scoring algorithms to extracted food access indicators, generating numeric scores that quantify overall food access levels for use in risk assessment calculations by the Predictive Analytics Engine 240.

In some embodiments, the Social Determinants of Health Module 230 may parse SDOH data to extract additional factors beyond housing status, employment status, and food access metrics. The SDOH data may further comprise at least one of transportation access, income level, or education status. The Social Determinants of Health Module 230 may extract transportation access by identifying transportation-related fields indicating availability of personal vehicles, access to public transportation, proximity to transit routes, and transportation barriers affecting access to healthcare, employment, or social services. The Social Determinants of Health Module 230 may extract income level by identifying income-related fields indicating household income amounts, income sources, income stability, and eligibility for income-based assistance programs. The Social Determinants of Health Module 230 may extract education status by identifying education-related fields indicating highest education level completed, current enrollment in educational programs, educational goals, and barriers to educational attainment.

At step 550, SDOH data is associated with an individual record in the centralized database. This operation is performed by the Social Determinants of Health Module 230, which establishes linkages between extracted SDOH data and existing individual records stored in the Data Repository 280. In some embodiments, the Social Determinants of Health Module 230 may associate SDOH data with individual records by identifying unique individual identifiers included in received SDOH data submissions, querying the Data Repository 280 via the Database Engine 270 to locate existing individual records matching identified identifiers, and establishing associations between extracted SDOH data elements and located individual records.

The Social Determinants of Health Module 230 may identify individual identifiers by extracting identifier values from designated fields in received SDOH data submissions. In some embodiments, individual identifiers may include system-generated unique identifiers assigned when individuals are first registered in the care coordination platform, government-issued identifiers such as social security numbers when authorized for use, healthcare identifiers such as medical record numbers from external EHR systems 285, or demographic-based identifiers constructed from combinations of name, date of birth, and other identifying information. The Social Determinants of Health Module 230 may apply identifier matching algorithms to locate existing individual records when exact identifier matches are not found, using probabilistic matching techniques that compare multiple identifying attributes to determine likely matches.

In some embodiments, the Social Determinants of Health Module 230 may create new individual records when no existing record matches the individual identified in received SDOH data submissions. The Social Determinants of Health Module 230 may create new records by generating unique system identifiers for new individuals, populating new records with identifying information extracted from SDOH data submissions, and storing new records in the Data Repository 280 via the Database Engine 270. The Social Determinants of Health Module 230 may flag newly created records for review by authorized users to verify that new records do not represent duplicate entries for individuals already registered under different identifiers.

The Social Determinants of Health Module 230 may establish associations between SDOH data and individual records by creating relational links between SDOH data records and individual records in the Data Repository 280. In some embodiments, the Social Determinants of Health Module 230 may implement associations using foreign key relationships where SDOH data records contain individual identifier fields that reference primary keys in individual records tables. The Social Determinants of Health Module 230 may also implement associations using junction tables that store mappings between SDOH data record identifiers and individual record identifiers, enabling flexible many-to-many relationships when individuals have multiple SDOH assessments over time.

At step 560, SDOH data is stored in the centralized database via the database engine. This operation is performed by the Social Determinants of Health Module 230 in coordination with the Database Engine 270. In some embodiments, the Social Determinants of Health Module 230 may store SDOH data in the Data Repository 280 by formatting extracted and associated SDOH data into database record structures, generating database commands to insert or update SDOH records, transmitting generated commands to the Database Engine 270, and receiving confirmation responses indicating successful storage operations.

The Social Determinants of Health Module 230 may format SDOH data for storage by mapping extracted data elements to corresponding database fields, converting data types as required by database schema definitions, applying data transformation rules to standardize values across different data sources, and generating complete record structures ready for database insertion. In some embodiments, the Social Determinants of Health Module 230 may generate SQL INSERT statements for new SDOH records by specifying target table names, listing field names for populated fields, and providing corresponding values for each field. The Social Determinants of Health Module 230 may generate SQL UPDATE statements for existing SDOH records by specifying target table names, listing field names and new values for modified fields, and specifying WHERE clauses that identify records to be updated based on unique identifiers.

The Database Engine 270 may execute received storage commands by parsing command syntax, validating data values against table constraints including data type constraints, not-null constraints, and foreign key constraints, acquiring necessary database locks to prevent concurrent modification conflicts, executing insert or update operations on target tables, committing completed transactions, and releasing acquired locks. In some embodiments, the Database Engine 270 may implement optimistic locking by checking version numbers or timestamps before committing updates to detect concurrent modifications, rejecting updates when concurrent modifications are detected, and requiring retry with refreshed data.

In some embodiments, the Social Determinants of Health Module 230 may maintain SDOH data history by storing each SDOH assessment as a separate record rather than overwriting previous assessments. This historical storage may enable tracking of SDOH changes over time, analysis of SDOH trends for individuals and populations, and comparison of current SDOH status with historical baselines. The Social Determinants of Health Module 230 may timestamp stored SDOH records with assessment dates indicating when SDOH data was collected, entry dates indicating when data was entered into the system, and modification dates indicating when records were last updated. The Social Determinants of Health Module 230 may generate audit records via the Audit Module 260 documenting SDOH data storage events including user identifiers for users who submitted or entered data, timestamps, and data values stored.

In some embodiments, the operations of steps 510 through 560 occur in response to SDOH data submissions from mobile devices or caseworker data entry, while in other embodiments they may be triggered by scheduled data collection campaigns or integration with external social service systems. The automated execution of these steps enables systematic collection and storage of SDOH data that captures non-clinical factors influencing health outcomes. By parsing received data to extract housing status, employment status, and food access metrics, the Social Determinants of Health Module 230 transforms unstructured or semi-structured input into standardized data elements that can be analyzed alongside healthcare data and shelter management data. The association of SDOH data with individual records in the centralized database enables the Predictive Analytics Engine 240 to incorporate SDOH factors into risk assessments, supporting identification of at-risk individuals based on combined analysis of clinical, housing, and social service data.

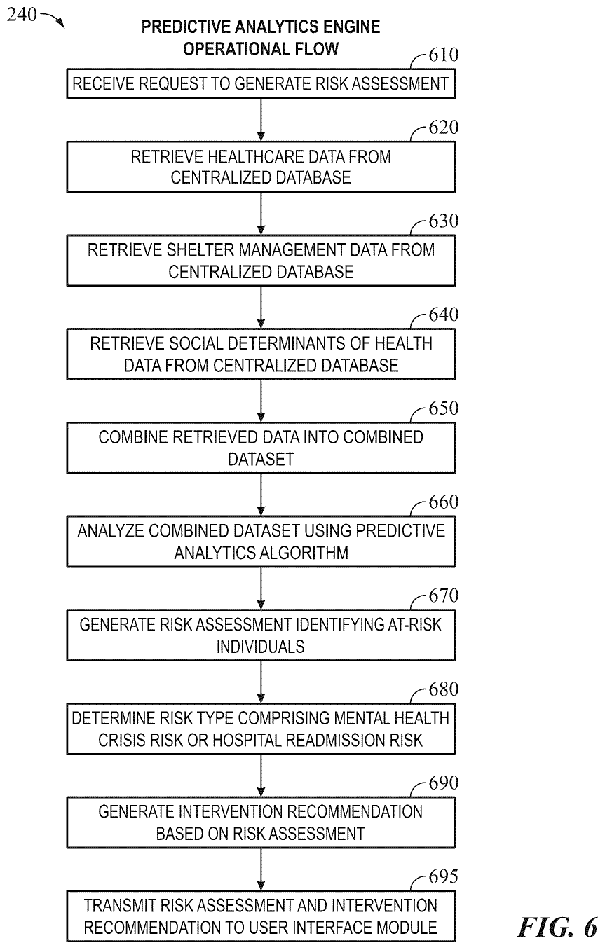

FIG. 6 is a flow diagram illustrating an exemplary operational sequence of the Predictive Analytics Engine 240 in accordance with certain embodiments. The sequence shown may be implemented as computer-executable instructions within the application program 200 operating on computing system 100 of FIG. 2, with specific operations performed by the modules depicted therein.

At step 610, a request to generate a risk assessment is received. This operation may be performed by the Predictive Analytics Engine 240, which receives risk assessment requests from other components of the application program 200 or from user computing devices 145 via the Communication and User Interface Module 250. In some embodiments, the request to generate a risk assessment may specify one or more individuals for whom risk assessments should be generated, risk types to be evaluated such as mental health crisis risk or hospital readmission risk, and output formats for generated risk assessments. The Predictive Analytics Engine 240 may parse received requests to extract request parameters including individual identifiers, risk type specifications, and formatting preferences.

In some embodiments, the Predictive Analytics Engine 240 may receive risk assessment requests through multiple channels. The Predictive Analytics Engine 240 may receive on-demand requests initiated by healthcare providers, shelter staff, or caseworkers seeking risk information for specific individuals they are currently serving. The Predictive Analytics Engine 240 may receive batch requests initiated by system administrators or automated processes seeking to generate risk assessments for defined populations such as all individuals currently residing in shelters, all individuals with recent emergency department visits, or all individuals with upcoming healthcare appointments. The Predictive Analytics Engine 240 may receive triggered requests initiated automatically when certain events occur, such as when new SDOH data is stored, when shelter status changes, or when clinical data is updated via the Electronic Health Record Interface Module 210.

The Predictive Analytics Engine 240 may validate received requests by confirming that requesting users have authorization to access risk assessment information for specified individuals, verifying that specified individuals exist in the Data Repository 280, and checking that requested risk types are supported by configured predictive models. In some embodiments, the Predictive Analytics Engine 240 may reject unauthorized requests and generate access denied responses, reject requests for non-existent individuals and generate not found responses, and reject requests for unsupported risk types and generate invalid request responses. The Predictive Analytics Engine 240 may accept validated requests and proceed with risk assessment generation.

At step 620, healthcare data is retrieved from the centralized database. This operation is performed by the Predictive Analytics Engine 240, which queries the Data Repository 280 via the Database Engine 270 to retrieve clinical information for individuals specified in the risk assessment request. In some embodiments, the Predictive Analytics Engine 240 may retrieve healthcare data by generating SQL SELECT statements specifying healthcare data tables, individual identifier filters matching individuals specified in the request, and field selections identifying specific healthcare data elements required for risk assessment calculations. The Predictive Analytics Engine 240 may transmit generated queries to the Database Engine 270 and receive result sets containing requested healthcare data.

In some embodiments, the healthcare data retrieved by the Predictive Analytics Engine 240 may comprise medical history data including diagnosis codes, diagnosis dates, and diagnosis status indicators; mental health diagnosis data including psychiatric diagnosis codes, treatment status, and current symptom severity; medication data including prescribed medications, dosages, and adherence indicators; healthcare utilization data including emergency department visits, hospitalizations, and outpatient appointments; and clinical assessment data including vital signs, laboratory results, and functional status measurements. The Predictive Analytics Engine 240 may retrieve healthcare data spanning defined time periods such as the past twelve months, the past three years, or all available history depending on configured data retrieval parameters and risk model requirements.

The Predictive Analytics Engine 240 may process retrieved healthcare data by parsing result sets returned by the Database Engine 270, extracting individual data elements from result records, converting extracted elements to data structures compatible with predictive analytics algorithms, and organizing extracted data by individual identifier for subsequent combination with data from other domains. In some embodiments, the Predictive Analytics Engine 240 may apply data cleaning operations to retrieved healthcare data by identifying and handling missing values, detecting and correcting data entry errors where possible, and flagging data quality issues for review when automatic correction is not feasible.

At step 630, shelter management data is retrieved from the centralized database. This operation is performed by the Predictive Analytics Engine 240, which queries the Data Repository 280 via the Database Engine 270 to retrieve shelter-related information for individuals specified in the risk assessment request. In some embodiments, the Predictive Analytics Engine 240 may retrieve shelter management data by generating SQL SELECT statements specifying shelter management data tables, individual identifier filters matching individuals specified in the request, and field selections identifying specific shelter data elements required for risk assessment calculations.

In some embodiments, the shelter management data retrieved by the Predictive Analytics Engine 240 may comprise current shelter status indicating whether individuals are currently residing in shelters, shelter history indicating previous shelter stays including admission dates, discharge dates, and shelter facility identifiers; shelter utilization patterns indicating frequency of shelter use, duration of shelter stays, and gaps between shelter episodes; and shelter service utilization indicating participation in services offered by shelter facilities such as case management, job training, or mental health counseling. The Predictive Analytics Engine 240 may retrieve shelter management data spanning defined time periods consistent with healthcare data retrieval parameters to enable temporal alignment of data from different domains.

The Predictive Analytics Engine 240 may process retrieved shelter management data by parsing result sets, extracting data elements, and organizing extracted data by individual identifier. In some embodiments, the Predictive Analytics Engine 240 may calculate derived shelter metrics from retrieved data, such as total shelter nights over defined periods, average shelter stay duration, number of shelter episodes, time since last shelter stay, and shelter stability indicators based on patterns of shelter utilization over time. These derived metrics may serve as input features for predictive analytics algorithms applied at step 660.

At step 640, social determinants of health data is retrieved from the centralized database. This operation is performed by the Predictive Analytics Engine 240, which queries the Data Repository 280 via the Database Engine 270 to retrieve SDOH information for individuals specified in the risk assessment request. In some embodiments, the Predictive Analytics Engine 240 may retrieve SDOH data by generating SQL SELECT statements specifying SDOH data tables, individual identifier filters matching individuals specified in the request, and field selections identifying specific SDOH data elements required for risk assessment calculations.

In some embodiments, the SDOH data retrieved by the Predictive Analytics Engine 240 may comprise housing status data including current housing classification and housing stability indicators; employment status data including current employment classification, employment duration, and income level; food access data including food security classification, meal frequency, and food assistance enrollment; and additional SDOH factors such as transportation access, education status, and social support indicators when available. The Predictive Analytics Engine 240 may retrieve the most recent SDOH assessment for each individual, or may retrieve historical SDOH assessments to enable analysis of SDOH trends over time.

The Predictive Analytics Engine 240 may process retrieved SDOH data by parsing result sets, extracting data elements, and organizing extracted data by individual identifier. In some embodiments, the Predictive Analytics Engine 240 may calculate derived SDOH metrics from retrieved data, such as SDOH composite scores combining multiple SDOH factors into unified indicators, SDOH change indicators reflecting improvements or deteriorations in SDOH status over time, and SDOH risk flags identifying specific SDOH factors that contribute disproportionately to adverse outcome risk. These derived metrics may serve as input features for predictive analytics algorithms.

At step 650, retrieved data is combined into a combined dataset. This operation is performed by the Predictive Analytics Engine 240, which integrates healthcare data retrieved at step 620, shelter management data retrieved at step 630, and SDOH data retrieved at step 640 into unified data structures suitable for predictive analytics processing. In some embodiments, the Predictive Analytics Engine 240 may be configured to analyze a combined dataset comprising the healthcare data, the shelter management data, and the SDOH data. The Predictive Analytics Engine 240 may combine retrieved data by matching records from different data domains based on common individual identifiers, joining matched records into unified individual profiles that span clinical, housing, and social service domains, and structuring joined records into tabular formats where each row represents an individual and each column represents a data element or derived metric.

The Predictive Analytics Engine 240 may handle data alignment challenges when combining data from multiple domains. In some embodiments, the Predictive Analytics Engine 240 may handle temporal misalignment by identifying the most recent data point from each domain for each individual, interpolating or carrying forward values when data collection dates differ across domains, and flagging individuals with temporal gaps between data points for potential data quality review. The Predictive Analytics Engine 240 may handle missing data by applying imputation techniques that estimate missing values based on available data, such as mean imputation, median imputation, or model-based imputation using machine learning algorithms trained on complete records.

In some embodiments, the Predictive Analytics Engine 240 may generate feature vectors for each individual in the combined dataset by selecting relevant data elements and derived metrics for inclusion in predictive model inputs, normalizing numeric features to standard scales, encoding categorical features as numeric values using techniques such as one-hot encoding or ordinal encoding, and assembling selected and transformed features into fixed-length vectors suitable for input to predictive algorithms. The combined dataset may comprise thousands or millions of feature vectors when batch risk assessments are generated for large populations, requiring efficient data processing techniques that leverage parallel computation capabilities of the computing system 100.

At step 660, the combined dataset is analyzed using a predictive analytics algorithm. This operation is performed by the Predictive Analytics Engine 240, which applies machine learning models or statistical algorithms to feature vectors in the combined dataset to generate risk predictions. In some embodiments, the Predictive Analytics Engine 240 may analyze the combined dataset using a predictive analytics algorithm by loading trained predictive models from model storage in the Data Repository 280, applying loaded models to feature vectors for each individual in the combined dataset, and generating prediction outputs indicating estimated risk levels for each individual.

In some embodiments, the predictive analytics algorithm applied by the Predictive Analytics Engine 240 may comprise machine learning classification models trained to distinguish between individuals who will experience adverse outcomes and individuals who will not experience adverse outcomes. Classification models may include logistic regression models that estimate outcome probabilities based on linear combinations of input features, random forest models that aggregate predictions from multiple decision trees trained on bootstrap samples, gradient boosting models that sequentially train decision trees to correct errors from previous trees, or neural network models that learn non-linear relationships between input features and outcomes through multiple layers of interconnected nodes.

The Predictive Analytics Engine 240 may apply predictive models by executing inference operations that process input feature vectors through model structures and generate output predictions. In some embodiments, the Predictive Analytics Engine 240 may execute logistic regression inference by computing dot products between feature vectors and learned coefficient vectors, applying sigmoid activation functions to computed dot products, and interpreting resulting values as estimated probabilities of adverse outcomes. The Predictive Analytics Engine 240 may execute random forest inference by passing feature vectors through each decision tree in the forest, collecting predictions from all trees, and aggregating collected predictions through majority voting or probability averaging. The Predictive Analytics Engine 240 may execute neural network inference by propagating feature vectors through network layers, applying activation functions at each layer, and extracting predictions from output layer nodes.

In some embodiments, the Predictive Analytics Engine 240 may apply multiple predictive models to evaluate different risk types or to generate ensemble predictions that combine outputs from multiple models. The Predictive Analytics Engine 240 may maintain separate models for mental health crisis risk prediction and hospital readmission risk prediction, each trained on historical data specific to the respective outcome type. The Predictive Analytics Engine 240 may also maintain ensemble configurations that combine predictions from multiple models using weighted averaging, stacking, or other ensemble techniques to improve prediction accuracy and robustness.

At step 670, a risk assessment is generated identifying at-risk individuals. This operation is performed by the Predictive Analytics Engine 240, which converts prediction outputs from step 660 into structured risk assessment records. In some embodiments, the Predictive Analytics Engine 240 may generate a risk assessment identifying at-risk individuals from the plurality of individuals by comparing prediction outputs to defined risk thresholds, classifying individuals with predictions exceeding thresholds as at-risk, and generating risk assessment records documenting classification results and supporting information.

The Predictive Analytics Engine 240 may apply risk thresholds by retrieving threshold values from configuration data stored in the Data Repository 280, comparing predicted risk probabilities or scores to retrieved threshold values, and assigning risk classifications based on comparison results. In some embodiments, the Predictive Analytics Engine 240 may apply multiple threshold levels to generate tiered risk classifications such as low risk, moderate risk, high risk, and critical risk, enabling prioritization of intervention resources toward individuals with highest risk levels. The Predictive Analytics Engine 240 may configure different threshold values for different risk types, reflecting different base rates and intervention priorities for mental health crisis risk versus hospital readmission risk.

In some embodiments, the Predictive Analytics Engine 240 may generate risk assessment records containing individual identifiers linking assessments to specific individuals, risk classification labels indicating assigned risk levels, predicted probability or score values underlying classifications, assessment timestamps indicating when risk assessments were generated, model identifiers indicating which predictive models were applied, and confidence indicators reflecting reliability of generated predictions. The Predictive Analytics Engine 240 may store generated risk assessment records in the Data Repository 280 via the Database Engine 270, enabling retrieval by other components of the application program 200 and historical tracking of risk assessment results over time.

At step 680, a risk type is determined comprising mental health crisis risk or hospital readmission risk. This operation is performed by the Predictive Analytics Engine 240, which evaluates prediction outputs to identify specific risk categories applicable to each at-risk individual. In some embodiments, the risk assessment generated by the Predictive Analytics Engine 240 may identify at least one of a mental health crisis risk or a hospital readmission risk. The Predictive Analytics Engine 240 may determine risk types by evaluating outputs from risk-type-specific predictive models applied at step 660, identifying which risk types have predictions exceeding applicable thresholds for each individual, and recording identified risk types in risk assessment records.

The Predictive Analytics Engine 240 may identify mental health crisis risk by evaluating predictions from models trained to predict outcomes such as psychiatric emergency department visits, psychiatric hospitalizations, suicide attempts, or other mental health crisis events. In some embodiments, the Predictive Analytics Engine 240 may evaluate combinations of mental health diagnosis data, shelter stay patterns, medication adherence indicators, recent service utilization, and SDOH factors that correlate with historical mental health crisis events. The Predictive Analytics Engine 240 may weight different predictive factors based on their relative importance as determined during model training, generating composite mental health crisis risk scores that reflect overall crisis likelihood.

The Predictive Analytics Engine 240 may identify hospital readmission risk by evaluating predictions from models trained to predict outcomes such as thirty-day hospital readmissions, emergency department visits within defined periods following hospital discharge, or other healthcare utilization events indicating care failures. In some embodiments, the Predictive Analytics Engine 240 may evaluate combinations of medical history, recent hospitalization data, housing instability indicators, social support factors, and medication adherence that correlate with historical readmission events. The Predictive Analytics Engine 240 may apply clinically validated readmission risk models or custom models trained on population-specific data to generate hospital readmission risk predictions.

In some embodiments, the Predictive Analytics Engine 240 may determine that individuals are at risk for multiple risk types simultaneously, generating risk assessment records that document all applicable risk types and associated prediction values. The Predictive Analytics Engine 240 may prioritize identified risk types based on severity, imminence, or intervention feasibility, enabling care coordinators to focus attention on highest-priority risks when multiple risks are present.

At step 690, an intervention recommendation is generated based on the risk assessment. This operation is performed by the Predictive Analytics Engine 240, which analyzes risk assessment results to determine appropriate intervention actions. In some embodiments, the Predictive Analytics Engine 240 may be further configured to generate an intervention recommendation based on the risk assessment, wherein the intervention recommendation comprises at least one of a caseworker outreach notification, a mental health service referral, or a healthcare provider alert. The Predictive Analytics Engine 240 may generate intervention recommendations by mapping identified risk types and risk levels to recommended intervention actions based on configured recommendation rules stored in the Data Repository 280.

The Predictive Analytics Engine 240 may generate caseworker outreach notifications by identifying at-risk individuals who would benefit from direct caseworker contact based on risk assessment results and current service engagement status. In some embodiments, the Predictive Analytics Engine 240 may generate caseworker outreach notifications for individuals with elevated risk who have not had recent caseworker contact, individuals whose SDOH status has deteriorated since their last assessment, or individuals who have recently experienced shelter status changes. The Predictive Analytics Engine 240 may format caseworker outreach notifications containing individual identifying information, risk assessment summaries, recommended outreach actions, and priority indicators reflecting urgency levels.

The Predictive Analytics Engine 240 may generate mental health service referrals by identifying at-risk individuals with elevated mental health crisis risk who are not currently engaged in appropriate mental health services. In some embodiments, the Predictive Analytics Engine 240 may generate mental health service referrals containing individual identifying information, mental health risk assessment details, recommended service types such as outpatient counseling, crisis intervention, or psychiatric evaluation, and suggested provider information based on individual location, insurance status, and service preferences when available. The Predictive Analytics Engine 240 may identify appropriate mental health service providers by querying provider directories stored in the Data Repository 280 or accessed via external service APIs.

The Predictive Analytics Engine 240 may generate healthcare provider alerts by identifying at-risk individuals with elevated hospital readmission risk or other healthcare-related risks who have upcoming healthcare appointments or active care relationships. In some embodiments, the Predictive Analytics Engine 240 may generate healthcare provider alerts containing individual identifying information, risk assessment summaries, relevant clinical and SDOH information that may inform care decisions, and recommended preventive interventions such as medication reconciliation, care plan review, or social services referral. The Predictive Analytics Engine 240 may route healthcare provider alerts to appropriate providers based on individual care team assignments documented in healthcare data retrieved from the Data Repository 280 or from external EHR systems 285.

In some embodiments, the Predictive Analytics Engine 240 may generate multiple intervention recommendations for individuals with multiple identified risk types or high-severity risk levels. The Predictive Analytics Engine 240 may prioritize generated recommendations based on risk severity, intervention urgency, and resource availability, enabling care coordinators to focus limited intervention resources on highest-impact actions. The Predictive Analytics Engine 240 may also generate recommendations for population-level interventions when batch risk assessments identify patterns affecting multiple individuals, such as recommendations to increase shelter capacity, expand mental health service availability, or implement targeted outreach programs.

At step 695, the risk assessment and intervention recommendation are transmitted to the user interface module. This operation is performed by the Predictive Analytics Engine 240, which delivers generated risk assessments and intervention recommendations to the Communication and User Interface Module 250 for presentation to appropriate users. In some embodiments, the Predictive Analytics Engine 240 may transmit risk assessment and intervention recommendation to the Communication and User Interface Module 250 by formatting generated outputs according to display specifications, routing formatted outputs to appropriate user interface components, and triggering notification delivery to relevant users.

The Predictive Analytics Engine 240 may format outputs for transmission by serializing risk assessment records and intervention recommendation records into data interchange formats such as JSON, packaging serialized records with metadata indicating target users and display contexts, and transmitting packaged outputs to the Communication and User Interface Module 250 via internal method calls or message queues. In some embodiments, the Communication and User Interface Module 250 may receive transmitted outputs, determine which users should receive risk assessment notifications based on user roles and individual assignments, generate notification messages containing relevant risk assessment information, and deliver generated notifications to user computing devices 145 via the network 190.

In some embodiments, the Communication and User Interface Module 250 may display transmitted risk assessments in dashboard views accessible to healthcare providers, shelter staff, and caseworkers. Dashboard displays may present risk assessment summaries showing at-risk individual counts by risk type and risk level, individual risk assessment details showing specific risk factors and intervention recommendations for selected individuals, and trend visualizations showing changes in population risk levels over time. The Communication and User Interface Module 250 may enable users to acknowledge intervention recommendations, document intervention actions taken, and update individual records based on intervention outcomes.

In some embodiments, the Predictive Analytics Engine 240 may generate audit records via the Audit Module 260 documenting risk assessment generation events including request parameters, data retrieved, models applied, predictions generated, risk classifications assigned, and intervention recommendations produced. These audit records may support quality assurance review of risk assessment processes, regulatory compliance documentation, and continuous improvement of predictive model performance.

In some embodiments, the operations of steps 610 through 695 occur automatically in response to risk assessment requests, data update events, or scheduled batch processing, while in other embodiments they may be initiated on-demand by authorized users seeking risk information for specific individuals or populations. The automated execution of these steps enables proactive identification of at-risk individuals based on integrated analysis of healthcare data, shelter management data, and SDOH data that spans traditionally fragmented service domains. By analyzing combined datasets using predictive analytics algorithms, the Predictive Analytics Engine 240 identifies risk patterns that would be difficult or impossible to detect through manual review of individual data sources. The generation of intervention recommendations translates risk identification into actionable guidance for healthcare providers, shelter staff, and caseworkers, enabling coordinated responses that address identified risks before adverse events occur.

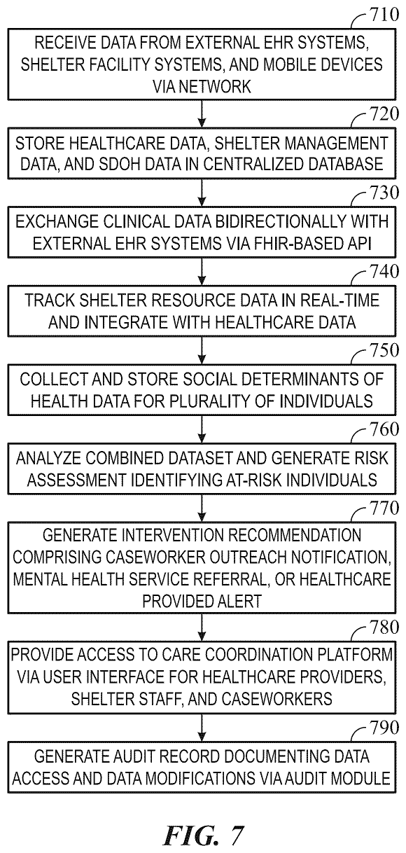

FIG. 7 is a flow diagram illustrating an exemplary end-to-end system operational flow in accordance with certain embodiments. The sequence shown may be implemented as computer-executable instructions within the application program 200 operating on computing system 100 of FIG. 2, demonstrating the integrated operation of multiple modules to coordinate healthcare services and social services across distributed systems and user populations.

At step 710, data is received from external EHR systems, shelter facility systems, and mobile devices via the network. This operation may be performed by multiple modules of the application program 200 operating in coordination to receive data from distributed external sources. In some embodiments, the Electronic Health Record Interface Module 210 may receive healthcare data from external EHR systems 285 via the network 190 by establishing connections using FHIR-based APIs, transmitting data requests, and receiving response messages containing clinical data as described with reference to FIG. 3. The Shelter Management Module 220 may receive shelter resource data from shelter facility systems 290 via the network 190 by establishing connections using configured APIs or data exchange protocols, transmitting data queries, and receiving response messages containing bed availability, intake status, and food service availability data as described with reference to FIG. 4. The Social Determinants of Health Module 230 may receive SDOH data from user computing devices 145 including mobile devices via the network 190 by receiving form submissions containing housing status, employment status, food access metrics, and additional SDOH factors as described with reference to FIG. 5.

In some embodiments, data reception at step 710 may occur continuously as external systems transmit updates, periodically according to configured synchronization schedules, or on-demand in response to specific data requests from other components of the application program 200. The computing system 100 may process data received from multiple external sources concurrently, leveraging multi-threaded or distributed processing capabilities to handle high volumes of incoming data without processing bottlenecks. The application program 200 may implement data validation and quality checks on received data to identify and handle malformed data, duplicate submissions, or data that fails integrity constraints before proceeding to storage operations.

At step 720, healthcare data, shelter management data, and SDOH data are stored in the centralized database. This operation is performed by the Database Engine 270, which receives data from the Electronic Health Record Interface Module 210, the Shelter Management Module 220, and the Social Determinants of Health Module 230 and stores received data in the Data Repository 280. In some embodiments, the Database Engine 270 may store healthcare data by executing SQL INSERT or UPDATE statements that persist clinical data including medical history, mental health diagnosis data, medication information, and healthcare utilization records in healthcare data tables. The Database Engine 270 may store shelter management data by executing SQL INSERT or UPDATE statements that persist shelter resource data including bed availability, intake status, and food service availability in shelter management data tables. The Database Engine 270 may store SDOH data by executing SQL INSERT or UPDATE statements that persist social determinants of health data including housing status, employment status, and food access metrics in SDOH data tables.

The Database Engine 270 may implement transaction management to ensure data consistency when storing data from multiple sources. In some embodiments, the Database Engine 270 may group related storage operations into atomic transactions, commit transactions when all operations complete successfully, and roll back transactions when any operation fails to prevent partial data updates. The Database Engine 270 may maintain referential integrity between data tables by enforcing foreign key constraints that require referenced records to exist before referencing records can be inserted. The Database Engine 270 may also maintain data indexes that enable efficient retrieval of stored data by the Predictive Analytics Engine 240 and other components requiring data access.

At step 730, clinical data is exchanged bidirectionally with external EHR systems via a FHIR-based API. This operation is performed by the Electronic Health Record Interface Module 210, which maintains bidirectional data exchange with external EHR systems 285 as described with reference to FIG. 3. In some embodiments, the Electronic Health Record Interface Module 210 may retrieve clinical data from external EHR systems 285 by transmitting FHIR-formatted data requests and receiving response messages containing requested clinical information. The Electronic Health Record Interface Module 210 may transmit updated data to external EHR systems 285 by formatting data according to FHIR specifications and transmitting formatted data via established API connections.

In some embodiments, the bidirectional data exchange may enable the care coordination platform to serve as an integration hub that synchronizes clinical information across multiple external EHR systems 285 that do not directly communicate with each other. The Electronic Health Record Interface Module 210 may retrieve clinical data from one external EHR system 285, store retrieved data in the Data Repository 280, and transmit relevant portions of stored data to other external EHR systems 285 that serve the same individuals. This integration capability may enable care coordination across organizational boundaries where healthcare providers use different EHR platforms that lack native interoperability.

The Electronic Health Record Interface Module 210 may implement conflict resolution when bidirectional data exchange results in conflicting data values. In some embodiments, the Electronic Health Record Interface Module 210 may apply timestamp-based conflict resolution that retains the most recently updated value, source-priority conflict resolution that prefers values from designated authoritative sources, or manual conflict resolution that flags conflicts for review by authorized users. The Electronic Health Record Interface Module 210 may maintain data provenance records that document the source system and timestamp for each data element, enabling traceability of data origins and change history.

At step 740, shelter resource data is tracked in real-time and integrated with healthcare data. This operation is performed by the Shelter Management Module 220, which maintains current shelter resource information and establishes linkages with healthcare data as described with reference to FIG. 4. In some embodiments, the Shelter Management Module 220 may track shelter resource data in real-time by receiving push notifications from shelter facility systems 290 when resource availability changes, polling shelter facility systems 290 at configured intervals to detect changes, or maintaining streaming connections that receive continuous updates. The Shelter Management Module 220 may update the Data Repository 280 via the Database Engine 270 when changes are detected, ensuring that stored shelter resource data reflects current conditions at shelter facilities.

In some embodiments, the Shelter Management Module 220 may integrate shelter resource data with healthcare data by establishing relational links between shelter records and healthcare records for the same individuals in the Data Repository 280. This integration may enable the Predictive Analytics Engine 240 to analyze combined data spanning clinical and housing domains without requiring separate data retrieval and manual joining operations. The integration may also enable the Communication and User Interface Module 250 to display shelter status alongside clinical information in unified individual profiles accessible to healthcare providers, shelter staff, and caseworkers.

The real-time tracking and integration capabilities may enable the care coordination platform to respond rapidly to changes in individual circumstances. In some embodiments, when an individual's shelter status changes from sheltered to unsheltered, the Shelter Management Module 220 may update the Data Repository 280, trigger re-evaluation of risk assessments by the Predictive Analytics Engine 240, and generate notifications to relevant caseworkers via the Communication and User Interface Module 250. This rapid response capability may enable proactive intervention when individuals experience housing instability that could lead to adverse health outcomes.

At step 750, social determinants of health data is collected and stored for a plurality of individuals. This operation is performed by the Social Determinants of Health Module 230, which receives, processes, and stores SDOH data as described with reference to FIG. 5. In some embodiments, the Social Determinants of Health Module 230 may collect SDOH data via mobile device input by receiving form submissions from caseworkers conducting field assessments, or via caseworker data entry by receiving form submissions from caseworkers entering data through web-based interfaces. The Social Determinants of Health Module 230 may parse received data to extract housing status, employment status, and food access metrics, associate extracted data with individual records, and store associated data in the Data Repository 280 via the Database Engine 270.

In some embodiments, the Social Determinants of Health Module 230 may collect SDOH data for a plurality of individuals through coordinated data collection campaigns. The Social Determinants of Health Module 230 may generate data collection assignments that identify individuals requiring SDOH assessments, distribute assignments to caseworkers via the Communication and User Interface Module 250, track assignment completion status, and aggregate collected data from multiple caseworkers into unified population datasets. This coordinated collection capability may enable systematic SDOH data gathering across large populations served by multiple caseworkers operating in distributed locations.

The SDOH data collected at step 750 may enable the care coordination platform to incorporate non-clinical factors into care coordination workflows. In some embodiments, the collected SDOH data may inform risk assessments generated by the Predictive Analytics Engine 240 by providing indicators of housing instability, employment barriers, food insecurity, and other factors that correlate with adverse health outcomes. The collected SDOH data may also inform intervention planning by identifying specific social service needs that should be addressed alongside clinical needs.

At step 760, the combined dataset is analyzed and a risk assessment is generated identifying at-risk individuals. This operation is performed by the Predictive Analytics Engine 240, which retrieves data from multiple domains, combines retrieved data into unified datasets, applies predictive analytics algorithms, and generates risk assessments as described with reference to FIG. 6. In some embodiments, the Predictive Analytics Engine 240 may analyze a combined dataset comprising the healthcare data, the shelter management data, and the SDOH data by retrieving data from the Data Repository 280 via the Database Engine 270, combining retrieved data based on common individual identifiers, and applying predictive analytics algorithms to combined data.

The Predictive Analytics Engine 240 may generate a risk assessment identifying at-risk individuals from the plurality of individuals by applying trained machine learning models or statistical algorithms to combined data, comparing prediction outputs to configured risk thresholds, and classifying individuals with predictions exceeding thresholds as at-risk. In some embodiments, the risk assessment may identify at least one of a mental health crisis risk or a hospital readmission risk based on evaluation of clinical indicators, shelter utilization patterns, and SDOH factors that correlate with respective adverse outcome types.

In some embodiments, the risk assessment generation at step 760 may occur on scheduled intervals to maintain current risk classifications for entire populations, on triggered events when individual data changes, or on demand when users request risk information for specific individuals. The Predictive Analytics Engine 240 may prioritize risk assessment generation based on data recency, with individuals having recently updated data receiving priority for risk re-evaluation over individuals with stable data.

At step 770, an intervention recommendation is generated comprising a caseworker outreach notification, a mental health service referral, or a healthcare provider alert. This operation is performed by the Predictive Analytics Engine 240, which analyzes risk assessment results and generates appropriate intervention recommendations as described with reference to FIG. 6. In some embodiments, the Predictive Analytics Engine 240 may generate an intervention recommendation based on the risk assessment by mapping identified risk types and risk levels to recommended intervention actions according to configured recommendation rules.

The intervention recommendation may comprise at least one of a caseworker outreach notification when at-risk individuals would benefit from direct caseworker contact, a mental health service referral when at-risk individuals have elevated mental health crisis risk and are not currently engaged in appropriate services, or a healthcare provider alert when at-risk individuals have elevated hospital readmission risk and have active healthcare relationships. In some embodiments, the Predictive Analytics Engine 240 may generate multiple intervention recommendations for individuals with multiple identified risk types or high-severity risk levels, prioritizing recommendations based on urgency and potential impact.

The intervention recommendations generated at step 770 may translate risk identification into actionable guidance for care coordination. In some embodiments, the generated recommendations may include specific suggested actions, recommended timeframes for intervention, relevant individual information that supports intervention planning, and indicators of recommendation priority. This actionable guidance may enable healthcare providers, shelter staff, and caseworkers to respond effectively to identified risks without requiring extensive manual analysis of risk assessment data.

At step 780, access to the care coordination platform is provided via a user interface for healthcare providers, shelter staff, and caseworkers. This operation is performed by the Communication and User Interface Module 250, which generates and delivers user interfaces to user computing devices 145 via the network 190. In some embodiments, the Communication and User Interface Module 250 may provide access to the care coordination platform for a plurality of user types comprising at least healthcare providers, shelter staff, and caseworkers by authenticating user credentials, determining user roles, and generating role-appropriate interfaces that display relevant information and functions.

The Communication and User Interface Module 250 may generate interfaces that display healthcare data, shelter management data, and SDOH data in consolidated views that enable users to assess individual needs comprehensively. In some embodiments, the Communication and User Interface Module 250 may display customizable dashboards presenting summary views of population risk levels, individual profiles spanning clinical and social service domains, and intervention tracking tools that document actions taken in response to recommendations. The Communication and User Interface Module 250 may provide mobile access to the care coordination platform via mobile computing devices, enabling caseworkers to access and enter data during field visits and enabling shelter staff to access information from any location within facilities.

In some embodiments, the Communication and User Interface Module 250 may deliver intervention recommendations generated at step 770 to appropriate users based on recommendation types and user assignments. The Communication and User Interface Module 250 may deliver caseworker outreach notifications to assigned caseworkers, mental health service referrals to mental health service coordinators, and healthcare provider alerts to assigned healthcare providers. The Communication and User Interface Module 250 may enable users to acknowledge received recommendations, document intervention actions, and record intervention outcomes for tracking and quality improvement purposes.

At step 790, an audit record is generated documenting data access and data modifications via the audit module. This operation is performed by the Audit Module 260, which receives logging requests from other components of the application program 200 and generates timestamped records documenting care coordination activities. In some embodiments, the Audit Module 260 may generate a record documenting data access and data modifications by each of the plurality of user types by capturing user identifiers, timestamps, accessed data elements, and modification details for each data access or modification event.

The Audit Module 260 may document data access events by recording which users accessed which data elements, when access occurred, and from which user computing devices 145 access was initiated. The Audit Module 260 may document data modification events by recording which users modified which data elements, what previous values were replaced, what new values were stored, and when modifications occurred. In some embodiments, the Audit Module 260 may implement tamper-evident logging by calculating and storing hash values for audit records, enabling subsequent verification that audit records have not been modified after initial storage.

In some embodiments, the audit records generated at step 790 may support regulatory compliance by documenting that data access and modifications comply with applicable privacy and security requirements. The audit records may support quality improvement by enabling analysis of care coordination activity patterns, identification of workflow inefficiencies, and tracking of intervention outcomes. The audit records may support accountability by documenting which users performed which actions, enabling investigation of data handling issues if they arise.

In some embodiments, the operations of steps 710 through 790 represent the complete end-to-end workflow for coordinating healthcare services and social services through the care coordination platform. The integrated operation of multiple modules enables the system to receive data from external EHR systems 285, shelter facility systems 290, and mobile devices; store healthcare data, shelter management data, and SDOH data in the centralized Data Repository 280; exchange clinical data bidirectionally with external EHR systems 285 via FHIR-based APIs; track shelter resource data in real-time and integrate with healthcare data; collect and store SDOH data for a plurality of individuals; analyze combined datasets and generate risk assessments identifying at-risk individuals; generate intervention recommendations comprising caseworker outreach notifications, mental health service referrals, or healthcare provider alerts; provide access to the care coordination platform for healthcare providers, shelter staff, and caseworkers; and generate audit records documenting data access and modifications.

This coordinated workflow may automate care coordination processes that traditionally required manual coordination across separate healthcare systems, shelter management systems, and social service databases. By integrating data from multiple domains into a unified platform with predictive analytics and intervention recommendation capabilities, the system may enable proactive identification of at-risk individuals and coordinated responses that address identified risks before adverse events occur. The automated data exchange, real-time tracking, and audit trail generation may reduce processing time, eliminate manual data transfer requirements, and ensure that complete documentation of care coordination activities is maintained for regulatory compliance and quality improvement purposes.

In this disclosure, the various embodiments are described with reference to the flowchart illustrations and/or block diagrams of methods, apparatus (systems), and computer program products. Those skilled in the art would understand that each block of the flowchart illustrations and/or block diagrams, and combinations of blocks in the flowchart illustrations and/or block diagrams, can be implemented by computer readable program instructions. The computer readable program instructions can be provided to a processor of a general purpose computer, special purpose computer, or other programmable data processing apparatus to produce a machine, such that the instructions, which execute via the processor of the computer or other programmable data processing apparatus, create means for implementing the functions or acts specified in the flowchart and/or block diagram block or blocks. The computer readable program instructions can be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks. The computer readable program instructions can be loaded onto a computer, other programmable data processing apparatus, or other device to cause a series of operational acts to be performed on the computer, other programmable apparatus, or other device to produce a computer implemented process, such that the instructions that execute on the computer, other programmable apparatus, or other device implement the functions or acts specified in the flowchart and/or block diagram block or blocks.

In this disclosure, the block diagrams in the Figures illustrate the architecture, functionality, and operation of possible implementations of systems, methods, and computer program products according to the various embodiments. Each block in the flowchart or block diagrams can represent a module, segment, or portion of instructions, which comprises one or more executable instructions for implementing the specified logical function(s). In some embodiments, the functions noted in the blocks can occur out of the order noted in the Figures. For example, two blocks shown in succession can, in fact, be executed concurrently or substantially concurrently, or the blocks can sometimes be executed in the reverse order, depending upon the functionality involved. In some embodiments, each block of the block diagrams and/or flowchart illustration, and combinations of blocks in the block diagrams and/or flowchart illustration, can be implemented by a special purpose hardware-based system that performs the specified functions or acts or carry out combinations of special purpose hardware and computer instructions.

In this disclosure, the subject matter has been described in the general context of computer-executable instructions of a computer program product running on a computer or computers, and those skilled in the art would recognize that this disclosure can be implemented in combination with other program modules. Generally, program modules include routines, programs, components, data structures, etc. that perform particular tasks and/or implement particular abstract data types. Those skilled in the art would appreciate that the computer-implemented methods disclosed herein can be practiced with other computer system configurations, including single-processor or multiprocessor computer systems, mini-computing devices, mainframe computers, as well as computers, hand-held computing devices (e.g., PDA, phone), microprocessor-based or programmable consumer or industrial electronics, and the like. The illustrated embodiments can be practiced in distributed computing environments where tasks are performed by remote processing devices that are linked through a communications network. Some embodiments of this disclosure can be practiced on a stand-alone computer. In a distributed computing environment, program modules can be located in both local and remote memory storage devices.

In this disclosure, the terms “component,” “system,” “platform,” “interface,” and the like, can refer to and/or include a computer-related entity or an entity related to an operational machine with one or more specific functionalities. The disclosed entities can be hardware, a combination of hardware and software, software, or software in execution. For example, a component can be a process running on a processor, a processor, an object, an executable, a thread of execution, a program, and/or a computer. By way of illustration, both an application running on a server and the server can be a component. One or more components can reside within a process and/or thread of execution and a component can be localized on one computer and/or distributed between two or more computers. In another example, respective components can execute from various computer readable media having various data structures stored thereon. The components can communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As another example, a component can be an apparatus that provides specific functionality through electronic components without mechanical parts, wherein the electronic components can include a processor or other means to execute software or firmware that confers at least in part the functionality of the electronic components. In some embodiments, a component can emulate an electronic component via a virtual machine, e.g., within a cloud computing system.

The phrase “application” as is used herein means software other than the operating system, such as Word processors, database managers, Internet browsers and the like. Each application generally has its own user interface, which allows a user to interact with a particular program. The user interface for most operating systems and applications is a graphical user interface (GUI), which uses graphical screen elements, such as windows (which are used to separate the screen into distinct work areas), icons (which are small images that represent computer resources, such as files), pull-down menus (which give a user a list of options), scroll bars (which allow a user to move up and down a window) and buttons (which can be “pushed” with a click of a mouse). A wide variety of applications is known to those in the art.

The phrases “Application Program Interface” and API as are used herein mean a set of commands, functions and/or protocols that computer programmers can use when building software for a specific operating system. The API allows programmers to use predefined functions to interact with an operating system, instead of writing them from scratch. Common computer operating systems, including Windows, Unix, and the Mac OS, usually provide an API for programmers. An API is also used by hardware devices that run software programs. The API generally makes a programmer's job easier, and it also benefits the end user since it generally ensures that all programs using the same API will have a similar user interface.

The phrases “computing device” or “central processing unit” as is used herein means a computer hardware component that executes individual commands of a computer software program. It reads program instructions from a main or secondary memory, and then executes the instructions one at a time until the program ends. During execution, the program may display information to an output device such as a monitor.

The term “execute” as is used herein in connection with a computer, console, server system or the like means to run, use, operate or carry out an instruction, code, software, program and/or the like.

In this disclosure, the descriptions of the various embodiments have been presented for purposes of illustration and are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein. Thus, the appended claims should be construed broadly, to include other variants and embodiments, which may be made by those skilled in the art.

It will be appreciated by persons skilled in the art that the present embodiment is not limited to what has been particularly shown and described hereinabove. A variety of modifications and variations are possible considering the above teachings without departing from the following claims.

I/We claim:

1. A computer-implemented integrated care coordination system for managing healthcare services and social services, the system comprising:
at least one computing device in operable communication with a network;
a server in operable communication with the at least one computing device over the network, the server configured to host a care coordination platform comprising:
a centralized database configured to store healthcare data, shelter management data, and social determinants of health data for a plurality of individuals;
an electronic health record interface module configured to exchange the healthcare data with at least one external electronic health record system via an application programming interface, wherein the electronic health record interface module is configured to retrieve clinical data from the at least one external electronic health record system and transmit updated data to the at least one external electronic health record system;
a shelter management module configured to track shelter resource data comprising at least bed availability and intake status for at least one shelter facility, wherein the shelter management module communicates the shelter resource data to the centralized database for integration with the healthcare data;
a social determinants of health module configured to collect and store social determinants of health data comprising at least housing status, employment status, and food access metrics for each of the plurality of individuals;
a predictive analytics engine configured to analyze a combined dataset comprising the healthcare data, the shelter management data, and the social determinants of health data to generate a risk assessment identifying at-risk individuals from the plurality of individuals; and
a user interface module configured to provide access to the care coordination platform for a plurality of user types comprising at least healthcare providers, shelter staff, and caseworkers.

2. The system of claim 1, wherein the application programming interface comprises a Fast Healthcare Interoperability Resources (FHIR)-based application programming interface configured to enable bidirectional data exchange between the care coordination platform and the at least one external electronic health record system.

3. The system of claim 1, wherein the clinical data retrieved by the electronic health record interface module comprises at least one of medical history or mental health diagnosis data.

4. The system of claim 1, wherein the electronic health record interface module is configured to exchange the healthcare data with the at least one external electronic health record system in real-time.

5. The system of claim 1, wherein the social determinants of health data further comprises at least one of transportation access, income level, or education status.

6. The system of claim 1, wherein the risk assessment generated by the predictive analytics engine identifies at least one of a mental health crisis risk or a hospital readmission risk.

7. The system of claim 1, wherein the predictive analytics engine is further configured to generate an intervention recommendation based on the risk assessment, wherein the intervention recommendation comprises at least one of a caseworker outreach notification, a mental health service referral, or a healthcare provider alert.

8. The system of claim 1, wherein the user interface module is further configured to provide mobile access to the care coordination platform via a mobile computing device.

9. The system of claim 1, wherein the server comprises a cloud-based server configured to synchronize data across a plurality of shelter facilities.

10. The system of claim 1, wherein the user interface module is configured to display a customizable dashboard presenting the healthcare data, the shelter management data, and the social determinants of health data.

11. The system of claim 1, wherein the social determinants of health module is configured to receive the social determinants of health data via at least one of a mobile device input or a caseworker data entry.

12. The system of claim 1, wherein the shelter management module is configured to track the shelter resource data in real-time.

13. The system of claim 1, wherein the shelter management module is further configured to track food service availability for the at least one shelter facility.

14. The system of claim 1, wherein the care coordination platform further comprises an audit module configured to generate a record documenting data access and data modifications by each of the plurality of user types.

15. The system of claim 8, wherein the user interface module is further configured to support offline data collection on the mobile computing device by caching data collection forms and individual records in local device storage, storing data entries in a synchronization queue during periods of network unavailability, and transmitting queued data entries to the server upon restoration of network connectivity.

16. The system of claim 1, wherein the care coordination platform further comprises a behavioral health scheduling module configured to maintain a directory of behavioral health service providers, match individuals with behavioral health providers based on clinical needs identified in the healthcare data, generate appointment requests, and track appointment attendance for incorporation into risk assessments generated by the predictive analytics engine.

17. The system of claim 1, wherein the care coordination platform is further configured to exchange data with at least one government healthcare program system, comprising retrieving at least one of eligibility indicators, coverage status data, or prior authorization status data for individuals from the at least one government healthcare program system, and transmitting at least one of service delivery records or referral status data to the at least one government healthcare program system.

18. The system of claim 7, wherein the care coordination platform is further configured to receive a referral submission identifying an individual and a requested service, automatically match the referral to at least one receiving facility based on resource availability and individual need, receive an acceptance or denial response from the at least one receiving facility, update a referral status record in the centralized database, and log a referral outcome upon completion of the referred service.

19. A computer-implemented method, executed by at least one processor of a computing device, for coordinating healthcare services and social services, the method comprising:
storing, via the computing device, healthcare data, shelter management data, and social determinants of health data for a plurality of individuals in a centralized database;
retrieving, via the computing device, clinical data from at least one external electronic health record system via an application programming interface;
transmitting, via the computing device, updated data to the at least one external electronic health record system via the application programming interface;
tracking, via the computing device, shelter resource data comprising at least bed availability and intake status for at least one shelter facility;
communicating, via the computing device, the shelter resource data to the centralized database for integration with the healthcare data;
collecting, via the computing device, social determinants of health data comprising at least housing status, employment status, and food access metrics for each of the plurality of individuals;
analyzing, via the computing device, a combined dataset comprising the healthcare data, the shelter management data, and the social determinants of health data;
generating, via the computing device, a risk assessment identifying at-risk individuals from the plurality of individuals based on the analyzing; and
providing, via the computing device, access to a user interface for a plurality of user types comprising at least healthcare providers, shelter staff, and caseworkers.

20. The method of claim 19, wherein the application programming interface comprises a Fast Healthcare Interoperability Resources (FHIR)-based application programming interface, and wherein retrieving the clinical data and transmitting the updated data occur in real-time.

21. The method of claim 19, further comprising generating, via the computing device, an intervention recommendation based on the risk assessment, wherein the intervention recommendation comprises at least one of a caseworker outreach notification, a mental health service referral, or a healthcare provider alert.

22. The method of claim 19, wherein analyzing the combined dataset comprises applying a predictive analytics algorithm to identify at least one of a mental health crisis risk or a hospital readmission risk for each of the plurality of individuals.

23. A software product comprising at least one computer-readable storage medium having application instructions stored on the at least one computer-readable storage medium, the application instructions executable by at least one processor to:
store healthcare data, shelter management data, and social determinants of health data for a plurality of individuals in a centralized database;
retrieve clinical data from at least one external electronic health record system via an application programming interface;
transmit updated data to the at least one external electronic health record system via the application programming interface;
track shelter resource data comprising at least bed availability and intake status for at least one shelter facility;
communicate the shelter resource data to the centralized database for integration with the healthcare data;
collect social determinants of health data comprising at least housing status, employment status, and food access metrics for each of the plurality of individuals;
analyze a combined dataset comprising the healthcare data, the shelter management data, and the social determinants of health data;
generate a risk assessment identifying at-risk individuals from the plurality of individuals based on the analysis; and
provide access to a user interface for a plurality of user types comprising at least healthcare providers, shelter staff, and caseworkers.

24. The software product of claim 23, wherein the application instructions are further executable to generate an intervention recommendation based on the risk assessment, wherein the intervention recommendation comprises at least one of a caseworker outreach notification, a mental health service referral, or a healthcare provider alert, and wherein the application programming interface comprises a Fast Healthcare Interoperability Resources (FHIR)-based application programming interface configured to enable bidirectional data exchange with the at least one external electronic health record system.
